(19) United States
(12) Patent Application Publication
(10) Pub. No.: US 2005/0129687 A1
(43) Pub. Date: Jun. 16, 2005

(54) Use of inhibitors of PACAP receptor activity for treatment of overactive bladder and pelvic floor pain syndrome

(75) Inventors: Margaret A. Vizzard, S. Burlington, VT (US); Peter Zvara, Stowe, VT (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: University of Vermont and State Agricultural College, Burlington, VT (US)

(21) Appl. No.: 10/980,985

(22) Filed: Nov. 3, 2004

Related U.S. Application Data
(60) Provisional application No. 60/516,862, filed on Nov. 3, 2003.

(51) Int. Cl. A61K 39/395; A61K 38/17

(52) U.S. Cl. 424/144.1; 514/12; 514/13; 514/14

(57) ABSTRACT

The present invention relates to the use of inhibitors of PACAP receptor activity for the treatment of pelvic floor pain syndrome and overactive bladder.

### RELATED APPLICATIONS

This application claims benefit under 35 U.S.C. 119(e) of U.S. provisional patent application 60/516,862 filed Nov. 03, 2003, the entire content of which is incorporated herein by reference.

### FEDERALLY SPONSORED RESEARCH

This invention was made with Government support under NIH-NIDDK Grant # KO1 DK02911-01A1, NIH-NIDDK and Woman's Health Initiative Grant # R29 DK51369 and renewed as 1 RO1 DK051369. The Government may have certain rights to this invention.

### FIELD OF INVENTION

This invention relates to methods of use of inhibitors of PACAP receptor activity for treating overactive bladder and pelvic floor pain syndrome.

### BACKGROUND OF INVENTION

Pituitary adenylate cyclase-activating polypeptide (PACAP) is a polypeptide hormone that stimulates adenylate cyclase in pituitary cells. Two forms of PACAP, PACAP-38 and PACAP-27 are known, and are equally potent in stimulating adenylate cyclase in pituitary cells. PACAP-38 contains 38 amino acid residues, whereas PACAP-27 carries the N-terminal 27 residues of PACAP-38. PACAP is a neuropeptide and is a member of a superfamily that includes several regulatory peptides, e.g., VIP, secretin and glucagon. PACAP is present, not only in various areas of the central nervous system, including the hypothalamus, posterior pituitary, cerebral cortex, and hippocampus, but also in peripheral tissues, such as the testis, adrenal gland, and the gut. At least two types of binding sites have been reported for PACAP in mammalian tissues, type I and type II, further divided into subtypes (PAC1, VPAC1, VPAC2). Type I receptors selectively recognize PACAP much more potently than VIP, but type II receptors display similar high affinity for PACAP-27, PACAP-38 and VIP. Type I PACAP receptors (PACI) are abundant in the CNS whereas the amount of type II receptors is low. Type II (VIP-PACAP) receptors almost exclusively interact with adenylate cyclase. At least two effector systems, however, exist for type I PACAP-preferring receptors. It appears that type I receptors stimulate both adenylate cyclase and phospholipase C, this coupling to dual signaling cascades involving interactions with G proteins of the Gₛ and G_{q} types. The functions of PACAP receptors, their agonists and antagonists have been studied. For example, U.S. Pat. No. 6,043,224 describes the use of PACAP antagonists for modulating expression production or formulation of APP myeloid precursor protein. It has also been shown that PACAP6-38 partially but significantly inhibited inhibitory-NANC or non-andrenergic, non-cholinergic relaxation in tracheal muscles in cats (Yoshida, M. et al., European Journal of Pharmacology, 2000). U.S. Pat. No. 6,469,055 describes the use of PACAP antagonists in APP and GFAP synthesis in astrocytes, and U.S. Pat. No. 6,184,284 claims the use of PACAP antagonists in treatments of Alzheimer's Disease.

### SUMMARY OF INVENTION

It has now been discovered that inhibitors of PACAP receptor activity can be used for treating pelvic floor pain syndrome and overactive bladder. One aspect of the invention relates to methods for treating pelvic floor pain syndrome in a subject by administering to the subject an inhibitor of PACAP receptor activity in an amount effective to reduce or eliminate the pelvic floor pain syndrome. In certain embodiments the pelvic floor pain syndrome is associated with a condition selected from the group consisting of: overactive bladder, painful bladder syndrome or interstitial cystitis, outlet obstruction, benign prostatic hyperplasia, detrusor smooth muscle, bladder afferents, neurogenic bladder, bacterial and non-bacterial infections, and incontinence.

Another aspect of the invention relates to a method for treating overactive bladder disorder in a subject by administering to the subject an inhibitor of PACAP receptor activity in an amount effective to reduce or eliminate the overactive bladder disorder. In certain embodiments the overactive bladder disorder is associated with a condition selected from the group consisting of: overactive bladder, interstitial cystitis, outlet obstruction, benign prostatic hyperplasia, detrusor smooth muscle, bladder afferents, neurogenic bladder, bacterial and non-bacterial infections, and incontinence.

Another aspect of the invention relates to a method for modulating micturition frequency in a subject in a need thereof, by administering to the subject an inhibitor of PACAP receptor activity in an amount effective to reduce the micturition frequency.

In certain embodiments of the invention the inhibitor of PACAP receptor activity is a PACAP antagonist. In preferred embodiments of the invention the PACAP antagonist is selected from the list comprised of: PACAP6-38, PACAP6-27 or a mixture thereof.

In one aspect of the invention the inhibitor of PACAP receptor activity is an isolated short RNA nucleotide that has sequence correspondence to PACAP receptor mRNA and mediates RNA interference by directing cleavage of the PACAP receptor mRNA to which it corresponds.

In another aspect of the invention the inhibitor of PACAP receptor activity is an isolated short RNA nucleotide that has sequence correspondence to PACAP receptor mRNA and inactivates the PACAP receptor gene by transcriptional silencing.

In yet another aspect of the invention the inhibitor of PACAP receptor activity is an isolated short RNA nucleotide that has sequence correspondence to PACAP receptor mRNA and targets the PACAP receptor mRNA for degradation.

In certain embodiments of the invention the subject is a human.

An aspect of the invention relates to a method of administration of the inhibitor of PACAP receptor activity, wherein the inhibitor of PACAP receptor activity is administered in a pharmaceutically acceptable carrier. In certain embodiments, the pharmaceutically acceptable carrier is formulated for interstitial administration. In another embodiment, the inhibitor of PACAP receptor activity is formulated for administration by a needle. In certain embodiments the inhibitor of PACAP receptor activity is formulated for administration by intravesical catheter implant. In yet another embodiment, the inhibitor of PACAP receptor activity is formulated for administration by intrathecal catheter implant.

In certain embodiments of the invention the effective amount of the inhibitor of PACAP receptor activity is administered from once a day to once every other week.

According to one aspect the invention relates to a kit comprising packaging material and a formulation contained within the packaging material, wherein the formulation comprises an inhibitor of PACAP receptor activity, wherein the packaging material comprises a label or package insert that states that the formulation can be administered to a subject having pelvic floor syndrome in an amount, at a frequency, and for a duration effective to reduce or eliminate the pelvic floor syndrome.

In another aspect the invention relates to a kit comprising packaging material and a formulation contained within the packaging material, wherein the formulation comprises an inhibitor of PACAP receptor activity, wherein the packaging material comprises a label or package insert that states that the formulation can be administered to a subject having overactive bladder disorder in an amount, at a frequency, and for a duration effective to reduce or eliminate the pelvic floor syndrome.

In yet another aspect the invention relates to a kit comprising packaging material and a formulation contained within the packaging material, wherein the formulation comprises an inhibitor of PACAP receptor activity, wherein the packaging material comprises a label or package insert that states that the formulation can be administered to a subject in need of modulating micturition frequency in an amount, at a frequency, and for a duration effective to reduce or eliminate the micturition frequency.

Another aspect of the invention provides a method for treating a PACAP receptor mediated disorder in a subject, comprising administering to the subject a modulator of PACAP receptor activity in an amount effective to modulate the PACAP receptor activity. In certain embodiments the modulator of PACAP receptor activity is an inhibitor of PACAP receptor activity. In yet other embodiments the modulator of PACAP receptor activity is a stimulator of PACAP receptor activity. In certain embodiments the PACAP receptor mediated disorder is selected from the group consisting of: pelvic floor pain syndrome, overactive bladder, interstitial cystitis, outlet obstruction, benign prostatic hyperplasia, detrusor smooth muscle, bladder afferents, neurogenic bladder, bacterial and non-bacterial infections, and incontinence, interstitial cystitis, outlet obstruction, benign prostatic hyperplasia, detrusor smooth muscle, bladder afferents, neurogenic bladder, bacterial and non-bacterial infections, incontinence and any combination thereof.

Another aspect of the invention provides a method for modulating micturition frequency in a subject, comprising administering to the subject a modulator of PACAP receptor activity in an amount effective to modulate the PACAP receptor activity. In certain embodiments the modulator of PACAP receptor activity is an inhibitor of PACAP receptor activity. In yet other embodiments the modulator of PACAP receptor activity is a stimulator of PACAP receptor activity.

### BRIEF DESCRIPTION OF DRAWINGS

The present invention may be more easily and completely understood when taken in conjunction with the accompanying figures.

FIG. 1 shows the effect of PACAP6-38 on the micturition frequency in a rat with chronic CYP-induced cystitis.

FIG. 2 is a histogram that shows the effect of PACAP6-38 on Non-voiding bladder contractions per cycle (NVC/cycle).

FIG. 3 is a histogram that shows the effect of PACAP6-38 on the Intercontraction interval (ICI).

FIG. 4 shows the effects of intravesically delivered PACAP6-38 on the appearance of non-voiding bladder contractions and the intercontraction interval in a rat with acute CYP-induced cystitis.

FIG. 5 is a schematic representation of the experimental set up used.

FIG. 6 shows the effect of intrathecally administered PACAP6-38 on micturition pressures in a rat with chronic spinal cord injury (SCI).

FIG. 7 is a bar graph that depicts the effects of PACAP6-38 on intermicturition pressure, threshold pressure and micturition pressure.

FIG. 8 is a bar graph that shows the changes in PACAP expression in L1 spinal cord region following spinal cord injury (*p≦0.001).

FIG. 9 is a bar graph that shows the changes in PACAP receptor expression in L6 spinal cord region after spinal injury (*p≦0.001).

FIG. 10 is a bar graph that shows the number of PACAP receptor cell profiles in dorsal root ganglia after spinal cord injury (*p≦0.001).

FIG. 11 is a bar graph that shows the number of PACAP receptor cell profiles in urothelium and detrusor smooth muscle after spinal cord injury (*p≦0.001).

### DETAILED DESCRIPTION OF THE INVENTION

According to some aspects of the invention, compounds which bind to the PACAP receptor and inhibit the activation of the PACAP receptor are useful in the treatment of overactive bladder and pelvic floor pain syndrome. By preventing the activation of the PACAP receptor, the inhibitors modulate the neurochemical phenotype of bladder afferent neurons.

An “inhibitor of PACAP receptor activity” is any molecular species that prevents PACAP receptor activity. The PACAP receptor activity inhibitor may function by preventing the transcription of a PACAP receptor gene, preventing the processing or translation of a PACAP receptor mRNA or preventing the processing, trafficking, or activity of a PACAP receptor protein when administered in vivo or in vitro to a mammalian cell which is otherwise competent to express active PACAP receptor. Thus, for example, inhibitors of PACAP receptor activity include repressors which prevent induction and/or transcription of the PACAP receptor gene, antisense sequences which selectively bind to PACAP receptor DNA or RNA sequences and which prevent the transcription or translation of the PACAP receptor gene, competitive and non-competitive inhibitors of the activity of the PACAP receptor protein. For example, an antisense molecule can be an oligonucleotide of between 5-100, preferably 10-50, preferably about 30 nucleotides in length, and complementary to a portion of the mRNA sequence (including the coding sequence) of the PACAP receptor gene. An siRNA molecule is preferably a double-stranded RNA molecule with a 19 base pair double-stranded region (corresponding to a sequence on the target gene or mRNA) with a 2 base overhang at both ends (preferably a TT dimer overhang at each end). Such molecules are described in greater detail herein. The term “inhibitor of PACAP receptor activity” is not intended to embrace non-selective suppressors of all gene expression or protein synthesis, or general toxins. The inhibitor of PACAP receptor activity can be a PACAP receptor binding molecule or a PACAP receptor antisense molecule. Other types of inhibitors of PACAP receptor activity include peptides, antibodies, including fragments of antibodies, such as Fc, which selectively bind to and inhibit the activity of the PACAP receptor (collectively referred to herein as “peptide substrates”); and ribozymes which interfere with the transcription, processing, or translation of PACAP receptor mRNA.

In addition, small molecules, peptide or non-peptides which structurally mimic the natural substrates of the PACAP receptor, but which do not activate the receptor fall within the class of inhibitors of PACAP receptor activity. This class of compounds also includes molecular species which do not mimic the natural substrates of the PACAP receptor, but which interact with the substrate binding domain or other regions of the PACAP receptor, preventing its activity. The PACAP receptor binding molecules may be easily prepared or identified by those of ordinary skill in the art using routine experiments since the PACAP receptor is a known compound which has been described structurally in the prior art.

Thus an inhibitor of PACAP receptor activity maybe an agent, ligand, agonist, or antagonist that binds to or exhibits an affinity for the PACAP receptor (VPAC1, PAC1, or VPAC2) and does not activate the receptor. When PACAP receptors are activated the stimulated cells produce cyclic adenosine 3′,5′-monophosphate (referred to as “cyclic AMP” or “cAMP” in the specification), inositol-3-phosphate and Ca²⁺ in certain cases, and further show the phenotypes that result from such binding and signaling.

The inhibitors of PACAP receptor activity include, but are not limited to PACAP antagonists such as peptide substrates that bind to the receptor. Examples of peptide substrates useful according to the invention include: PACAP6-38, PACAP6-27 and mixtures thereof. PACAP6-38 (Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys SEQ. ID NO. 1) is a PAC1-specific receptor selective antagonist that is a truncated version of the PACAP peptide that is missing the first six amino acids of the carboxylic terminus. PACAP6-27 (Phe-Thr-Asp-Ser-Tyr-Ser-Arg-Tyr-Arg-Lys-GIn-Met-Ala-Val-Lys-Lys-Tyr-Leu-Ala-Ala-Val-Leu SEQ. ID NO. 2) is a selective PACAP antagonist that is a truncated version of the PACAP peptide. The truncations are on both the carboxy and the amino termini.

Other peptide substrates can easily be identified without undue experimentation in vitro based on their ability to bind or activate the PACAP receptor. To determine whether a peptide binds to the PACAP receptor any known binding assay may be employed. For example, in the case of a peptide that binds to the PACAP receptor the peptide may be immobilized on a surface and then contacted with a labeled PACAP receptor (or vice versa). The amount of PACAP receptor which interacts with the peptide or the amount which does not bind to the peptide may then be quantitated to determine whether the peptide binds to PACAP receptor. A surface having a known peptide that binds to PACAP receptor such as a monoclonal antibody immobilized thereto may serve as a positive control.

The activation of the PACAP receptor can be assayed by measuring the increase of intracellular cAMP concentration, increase of the inositol-3-phosphate concentration, or expansion of nervous processes. The following descriptions are non-limiting examples only. The activation of the PACAP receptor can be assayed by incubating the cells in medium (e.g., DMEM, HANKS) in the presence of such a peptide at a temperature of about 37° C. for several minutes (e.g., 10 minutes). The time course of production of cAMP, inositol-3-phosphate and Ca²⁺ can be detected by measuring methods well known in the art (Masmoudi et al, FASEB, 17:17-27 (2003)). For example, the following assays can be used for measuring the production of cAMP, inositol-3-phosphate matabolism and Ca²⁺ cellular concentration:

Primary cultures of rat cortical type 1 astrocytes are prepared as described in Papadopoulos et al., Endocrinology 129:1481-1488 (2002). Cerebral hemispheres from newborn Wistar rats are collected in DMEM/F12 (2/1; V/V) culture medium supplemented with 2 mM glutamine, 1% insulin, 5 mM HEPES, 0.4% glucose, and 1% of the antibiotic antimycotic solution. Tissues are dissociated mechanically with a syringe equipped with a 1 mm gauge needle and filtered through a 100 μm sieve (Falcon, Franklin Lakes, N.J.). Dissociated cells are resuspended in culture medium supplemented with 10% FCS and seeded at a density of 0.6×10⁶ cells/mL. To measure cAMP production and determine phosphoinositol, cells are seeded in 35 mm dishes (Dutscher). To measure intracellular calcium concentrations, astrocytes are cultured on 25 mm coverslips in 35 mm dishes. Cells are incubated at 37° C. in a humid atmosphere (5% CO2) and the medium is changed twice a week.

Measurement of cAMP may be carried out, for example, by the following protocol: Nine- to 12-day-old cultured cells are preincubated for 30 min with serum-free medium containing 100 μM IBMX. The cells are then incubated in the absence or presence of test substances. Incubation is stopped by removing the medium and adding 10% (w/v) ice-cold TCA. Cells are homogenized and centrifuged (14,000 g, 4° C., 10 min). The supernatant is washed three times with 1 mL water-saturated diethylether, dried, and reconstituted in RIA buffer (0.05 M sodium acetate, pH 5.8). The concentration of cAMP is measured by using a cAMP RIA kit (RPA 509; Amersham International). The pellets are used to measure protein concentration by the Lowry method.

Measurement of phosphoinositol metabolism may be carried out by the following procedure: Nine- to 12-day-old culture cells are incubated at 37° C. with 10 μCi/mL myo-[3H]inositol in glucose- and serum-free medium in the absence or presence of test substances. Incubation is stopped by removing the medium and adding 10% (W/V) ice-cold TCA. The cells are homogenized and centrifuged (13,000 g, 4° C., 10 min). The supernatant containing the phosphoinositols (IPs) is washed three times with 1 mL water-saturated diethylether neutralized with 10 μL of 1 M NaHCO₃. Free [³H]inositol and [³H]IPs are separated by anion exchange chromatography (on AG1-X8 resin mini columns, 100-200 mesh, formate form, Bio-Rad Laboratories, Richmond, Calif.) using distilled water and 0.8 M ammonium formate in 0.1 M formic acid, respectively. The radioactivity contained in each fraction is counted in a beta counter (LKB 1217 Rack Beta, EG and G Wallac, Evry, France). [³H]Polyphosphoinositides ([³H]PIPs) are extracted from the pellet with 500 μL of chloroform/methanol (2:1, v/v) and counted in a beta counter. The remaining pellets are used to measure protein concentration by the Lowry method.

Measurement of intracellular calcium concentration maybe made in the following manner: Five- to 7-day-old cultured cells are incubated at 37° C. for 45 min with 5 μM fluo-4 acetoxymethyl ester (fluo-4 AM) diluted in culture medium. Thereafter, the cells are washed with fresh medium. Astrocytes are mounted in a thermostatically controlled perfusion chamber maintained at 37° C. and continuously perfused with culture medium in the absence or presence of test substances. The cells are examined on a Noran OZ confocal laser scanning microscope (Noran Instrument, Middleton, Wis.) equipped with a krypton-argon laser (excitation wavelength: 488 nm) and the fluorescence emitted is recorded using a 500 nm long-pass filter. Images are recorded as a time series (512×480 pixels at 1 image per 532 ms) and data processing is carried out using Intervision Software (Noran Instrument).

A positive control for inhibition may be used by adding PACAP6-38 at the initiation of the reaction. In the positive control for inhibition, the PACAP receptor is not activated because the PACAP is inhibited from binding to the receptor. A control for activation may be used by allowing the reaction to proceed in the presence of PACAP and in the absence of antagonist or inhibitor of PACAP receptor activation. In the control for activation the PACAP receptor is activated because PACAP is allowed to bind to the receptor uninhibited. This type of activation assays also can be used to assess the relative inhibitory concentrations of a peptide and to identify those peptides which significantly inhibit PACAP receptor activation, e.g. by at least 75%. Other assays which are useful for determining whether a peptide which binds to PACAP receptor also inhibits PACAP receptor activation will be apparent to those of skill in the art, having read the present specification.

The inhibitor of PACAP receptor activity can be a specific PACAP receptor inhibitor. A “specific PACAP receptor inhibitor” as used herein is any molecular species which is a PACAP receptor inhibitor as defined above, but which specifically inhibits only one or more of VPAC1, PAC1, or VPAC2 receptors.

The “PACAP receptors” in the invention are membrane-bound proteins existing in various tissues. Since significant differences in functions of PACAP receptors are not observed among animal species PACAP receptors can be used regardless of their origin. Cells where PACAP receptors are expressed, methods for preparing the cells, and cDNA vectors for their expression are well known in the art (Pisgna et al., PNAS 90: 6245-6249 (1993), Sreedharan et al., Biochem. Biophys. Res. Commun. 193:546-553 (1993)). In brief, PACAP receptors can be prepared from rat brain tissue from which the cerebellum is removed by homogenization at 4° C. Following centrifugation (1,000×g, 10 minutes, 4° C.), the supernatant is centrifuged at 30,000×g for 45 minutes at 4° C. and the pellet is washed and resuspended in 50 mM Tris-HCl buffer. Other methods are known to those of ordinary skill in the art. The receptors mentioned in the invention include not only those disclosed in these literature references, but also those which are derived from other various mammals and can be prepared according to the description of these literature references. Examples of sequences of the PACAP receptors have been recorded under the following GENBANK acquisition numbers: SEQ ID NO: 1 gi|34398688|ref|NM_{—}001118.3|, SEQ ID NO: 2 gi|33188464|ref|NM_{—}003382.3|, SEQ ID NO: 3 [gi|15619005|ref|NM_{—}004624.21, SEQ ID NO: 4 gi|457562|dbj|D17516.1|, SEQ ID NO: 5 gi|1684934|gb|U18810.1|, and SEQ ID NO: 6 gi|550477|gb|L36566.1|.

The inhibitor of PACAP receptor activity is an isolated molecule. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

One aspect of the invention relates to the method of treating pelvic floor pain syndrome, overactive bladder disorder and/or modulating micturition frequency by using isolated short RNA that directs the sequence-specific degradation of PACAP mRNA through a process known as RNA interference (RNAi). The process is known to occur in a wide variety of organisms, including embryos of mammals and other vertebrates. It has been demonstrated that dsRNA is processed to RNA segments 21-23 nucleotides (nt) in length, and furthermore, that they mediate RNA interference in the absence of longer dsRNA. Thus, these 21-23 nt fragments are sequence-specific mediators of RNA degradation. This present invention encompasses the use of these fragments (or recombinantly produced or chemically synthesized oligonucleotides of the same or similar nature) to enable the targeting of PACAP mRNAs for degradation in mammalian cells useful in the therapeutic applications discussed herein.

The methods for design of the RNA's that mediate RNAi and the methods for transfection of the RNAs into cells and animals is well known in the art and are readilly commercially available (Verma N. K. et al, J. Clin. Pharm. Ther., 28(5):395-404(2004), Mello C. C. et al. Nature, 431(7006)338-42 (2004), Dykxhoorn D. M. et al., Nat. Rev. Mol. Cell Biol. 4(6):457-67 (2003) Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 μmol-scale RNA synthesis provides about 1 milligram of RNA, which is sufficient for 1000 transfection experiments using a 24-well tissue culture plate format.

The PACAP cDNA specific siRNA is designed by selecting a sequence that is not within 50-100 bp of the start codon and the termination codon, avoids intron regions, avoids streches of 4 or more bases such as AAAA, CCCC, avoids regions with GC content <30% or >60%, avoids repeats and low complex sequence, and it avoids single nucleotide polymorphism sites. The PACAP siRNA is designed by a search for a 23-nt sequence motif AA(N₁₉). If no suitable sequence is found, then a 23-nt sequence motif NA(N₂₁) and convert the 3′ end of the sense siRNA to TT. Alternatively, the PACAP siRNA can be designed by a search for NAR(N₁₇)YNN. The target sequence should have a GC content of around 50%. The siRNA targeted sequence is further evaluates using a BLAST homology search to avoid off target effects on other genes or sequences. Negative controls are designed by scrambling targeted siRNA sequences. The control RNA has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 which are hereby incorporated by reference in their entirety.

The methods described herein are used to identify or obtain RNA molecules that are useful as sequence-specific mediators of PACAP mRNA degradation and, thus, for inhibiting PACAP receptor activity. Expression of the PACAP receptor can be inhibited in humans in order to prevent the disease or condition from occurring, limit the extent to which it occurs or reverse it. The diseases and conditions that can be treated in this manner include but are not limited to pelvic floor pain syndrome and overactive bladder disorder. Using the PACAP sequence RNAs can be produced and tested for their ability to mediate RNAi in a cell, such as a human or other primate cell. Those RNA molecules shown to mediate RNAi can be tested, if desired, in an appropriate animal model to further assess their in vivo effectiveness.

The RNA molecules can be isolated using a number of techniques known to those of skill in the art. For example, gel electrophoresis can be used to separate RNAs from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate RNAs.

Any dsRNA can be used in the methods of the present invention, provided that it has sufficient homology to the PACAP receptor gene to mediate RNAi. The dsRNA for use in the present invention can correspond to the entire PACAP receptor gene or portion thereof. There is no upper limit on the length of the dsRNA that can be used. For example, the dsRNA can range from about 21 base pairs (bp) of the gene to the full length of the gene or more. In one embodiment, the dsRNA used in the methods of the present invention is about 1000 bp in length. In another embodiment, the dsRNA is about 500 bp in length. In yet another embodiment, the dsRNA is about 22 bp in length. In certain embodiments the preferred length of the RNA of the invention is 21 to 23 nucleotides.

The inhibitors of PACAP receptor activity of the invention also encompass antisense oligonucleotides that selectively bind to a PACAP receptor nucleic acid molecule, and dominant negative constructs used to reduce the expression of the PACAP receptor. Antisense oligonucleotides are useful, for example, for reducing the expression of PACAP receptor proteins in PACAP receptor expressing cells.

As used herein, the term “antisense oligonucleotide” or “antisense” describes an oligonucleotide which hybridizes under physiological conditions to DNA comprising a particular gene or to an RNA transcript of that gene and, thereby, inhibits the transcription of that gene and/or the translation of the mRNA. The antisense molecules are designed so as to hybridize with the target gene or target gene product and thereby, interfere with transcription or translation of the target mammalian cell gene. Those skilled in the art will recognize that the exact length of the antisense oligonucleotide and its degree of complementarity with its target will depend upon the specific target selected, including the sequence of the target and the particular bases which comprise that sequence. The antisense must be a unique fragment. A unique fragment is one that is a ‘signature’ for the larger nucleic acid. It, for example, is long enough to assure that its precise sequence is not found in molecules outside of the PACAP receptor gene. As will be recognized by those skilled in the art, the size of the unique fragment will depend upon its conservancy in the genetic code. Thus, some regions of the PACAP receptor gene will require longer segments to be unique while others will require only short segments, typically between 12 and 32 base pairs (e.g. 12, 13, 14, 15, 16, 17, 18, 19, 20, 21, 22, 23, 24, 25, 26, 27, 28, 29, 30, 31 and 32 bases long).

It is preferred that the antisense oligonucleotide be constructed and arranged so as to bind selectively with the target under physiological conditions, i.e., to hybridize substantially more to the target sequence than to any other sequence in the target cell under physiological conditions. Based upon the known sequence of a gene that is targeted for inhibition by antisense hybridization, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 7 and, more preferably, at least 15 consecutive bases which are complementary to the target. Most preferably, the antisense oligonucleotides comprise a complementary sequence of 20-30 bases. Although oligonucleotides may be chosen which are antisense to any region of the gene or RNA (e.g., mRNA) transcripts, in preferred embodiments the antisense oligonucleotides are complementary to 5′ sites, such as translation initiation, transcription initiation or promoter sites, that are upstream of the gene that is targeted for inhibition by the antisense oligonucleotides. In addition, 3′-untranslated regions may be targeted. Furthermore, 5′ or 3′ enhancers may be targeted. Targeting to mRNA splice sites has also been used in the art but may be less preferred if alternative mRNA splicing occurs. In at least some embodiments, the antisense is targeted, preferably, to sites in which mRNA secondary structure is not expected (see, e.g., Sainio et al., Cell Mol. Neurobiol., (1994) 14(5):439-457) and at which proteins are not expected to bind. The selective binding of the antisense oligonucleotide to a mammalian target cell nucleic acid effectively decreases or eliminates the transcription or translation of the mammalian target cell nucleic acid molecule.

The inhibitor of PACAP receptor activity may be isolated from natural sources or synthesized or produced by recombinant means. Methods for preparing or identifying molecules which bind to a particular target are well-known in the art. Molecular imprinting, for instance, may be used for the de novo construction of macro molecular structures, such as peptides, which bind to a particular molecule. See for example, Kenneth J. Shea, Molecular Imprinting of Synthetic Network Polymers: The De novo Synthesis of Molecular Binding In Catalytic Sites, Trip, to May 1994; Klaus, Mosbach, Molecular Imprinting, Trends in Biochem. Sci., 19(9), January 1994; and Wulff, G., In Polymeric Reagents and Catalysts (Ford, W.T., ed.) ACS Symposium Series No. 308, P. 186-230, Am. Chem. Soc. 1986. Binding peptides, such as antibodies, may easily be prepared by generating antibodies to the PACAP receptor or by screening libraries to identify peptides or other compounds which bind to the PACAP receptor.

Although the examples below are directed to a preferred embodiment of the invention, namely, PACAP peptide substrates and associated methods of use for inhibiting PACAP receptor activation, it should be understood that this description is illustrative only and is not intended to limit the scope of the instant invention. Any peptide or other molecule which binds to the PACAP receptor and inhibits activation of the PACAP receptor is encompassed by the methods of the invention. For example, the molecules of the invention include the peptide substrates, e.g. PACAP6-38, PACAP6-27 antibodies and functionally active fragments of antibodies and other small peptides exhibiting PACAP receptor specificity, as well as non-peptide compounds.

Thus, the inhibitors of the invention may be the peptide substrates, or non-peptide molecules. Methods are known for identifying other molecules other than those specifically exemplified in the specification. For instance, mimics of known binding peptides may also be prepared by known methods, such as (i) polymerization of functional monomers around a known binding peptide or the binding region of an antibody which also binds to the target (the template) that exhibits the desired activity; (ii) removal of the template molecule; and then (iii) polymerization of a second class of monomers in the void left by the template, to provide a new molecule which exhibits one or more desired properties which are similar to that of the template. In addition to preparing peptides, other binding molecules which have the same function as the binding peptide, such as polysaccharides, nucleotides, nucleoproteins, lipoproteins, carbohydrates, glycoproteins, steroids, lipids and other biologically-active material can also be prepared. Thus a template, such as a PACAP receptor binding antibody or a PACAP receptor substrate e.g. PACAP6-38 or PACAP6-27 can be used to identify PACAP receptor inhibitors. It is now routine to produce large numbers of inhibitors based on one or a few peptide sequences or sequence motifs. (See, e.g., Bromme, et al., Biochem. J. 315:85-89 (1996); Palmer, et al., J. Med. Chem. 38:3193-3196 (1995)). For example, since PACAP6-38 is known to inhibit the PACAP receptor activity, an inhibitor of the PACAP receptor may be chosen or designed as a modified polypeptide having structural or sequence similarity to PACAP6-38. Thus, a plurality of PACAP receptor inhibitors chosen or designed based on the sequence or structure of the preferred PACAP6-38, may be produced, tested for inhibitory activity, and sequentially modified to optimize or alter activity, stability, and/or specificity.

The method is useful for designing a wide variety of biological mimics that are more stable than the natural counterpart, because they are typically prepared by the free radical polymerization of functional monomers, resulting in a compound with a non-biodegradable backbone. Thus, the created molecules would have the same binding properties as the PACAP receptor substrate but be more stable in vivo, thus preventing the PACAP receptor from interacting with its native substrate. Other methods for designing such molecules include, for example, drug design based on structure activity relationships which require the synthesis and evaluation of a number of compounds and molecular modeling.

Alternatively, inhibitors of PACAP receptor activity can be identified from combinatorial libraries. Many types of combinatorial libraries have been described. For instance, U.S. Pat. No. 5,712,171 (which describes methods for constructing arrays of synthetic molecular constructs by forming a plurality of molecular constructs having the scaffold backbone of the chemical molecule and modifying at least one location on the molecule in a logically-ordered array); U.S. Pat. No. 5,962,412 (which describes methods for making polymers having specific physiochemical properties); and U.S. Pat. No. 5,962,736 (which describes specific arrayed compounds).

Identification of molecules useful according to the invention, also can be carried out utilizing a competition assay. If a molecule being tested competes with the PACAP6-38, PACAP or an anti-PACAP monoclonal antibody, as shown by a decrease in binding of the PACAP6-38, PACAP or the monoclonal antibody, then it is likely that the molecule, PACAP6-38, PACAP and the monoclonal antibody bind to the same, or a closely related, epitope.

In some embodiments the inhibitor of PACAP receptor activity is an antibody, or a functionally active antibody fragment, or a peptide substrate Antibodies are well known to those of ordinary skill in the science of immunology. As used herein, the term “antibody” means not only intact antibody molecules but also fragments of antibody molecules retaining PACAP receptor binding ability. Such fragments are also well known in the art and are regularly employed both in vitro and in vivo. In particular, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983)).

In one set of embodiments, the antibody useful according to the methods of the present invention is an intact, fully human anti-PACAP receptor monoclonal antibody in an isolated form or in a pharmaceutical preparation. The following is a description of a method for developing a monoclonal antibody that interacts with and inhibits the activation of PACAP receptor. PACAP receptor antibodies are also commercially available from a variety of sources for example: Research Diagnostics, Inc., Flanders, N.J.; Alpha Diagnostics International Inc., San Antonio, Tex.; and Phoenix Pharmaceuticals Inc., Mountain View, Calif. The description is exemplary and is provided for illustrative purposes only.

Murine monoclonal antibodies may be made by any of the methods known in the art utilizing PACAP receptor as an immunogen. An example of a method for producing murine monoclonals useful according to the invention is the following (including production of Receptor production of antibody to the Receptor and analysis):

Construction of the expression plasmids. The fragment of cDNA coding the putative extracellular N-terminal part (151 amino acid residues) of the PaCAP receptor may be amplified, for example, using the plasmid pNtPACAPR as template and the following primers: 5′CCG CTC GAG CAT ATG GCC AGA CTC CTG C 3′ Seq. ID. NO. 3 (sense); and 5′ GCG CTC GAG CGA CAG GTA GTA ATA ATC 3′ Seq. ID. NO. 4 (antisense).

The polymerase chain reaction (PCR) fragment is cloned into Pst I and Hind III sites of the vector pMAL-c2 (New England Biolabs, Beverly, Mass.) to yield the expression plasmid pMAL-PACAPr-N. After induction of the transformed E. coli CAG626 cells with 1 mM isopropyl-beta d-thiogalactopyranoside (IPTG) the fused protein is obtained consisting of the maltose-binding protein and the PACAP receptor's extracellular N-terminal part (Mal-PACAPr-N). The soluble fraction of the sonicated cell lysate is adsorbed on the amylose resin (New England Biolabs) column. The fused protein is eluted with 10 mM maltose solution according to the supplier's instruction.

The fragment from the plasmid pMAL-PACAP-N coding extracellular N-terminal part of the PACAP receptor including small part of pMAL-c2 vector (EcoRi-Pst1 fragment) is cloned into EcoRI-HindIII sites of the vector pET-28A (Novagen, Madison, Wis.) to yield the expression plasmid pET-PACAPr-N. The protein with His-tags on both N- and C-terminal is expressed after induction with 1 mM isopropyl-beta D-thiogalactopyranoside (IPTG). The tagged protein (PACAPr-N) may be found as inclusion bodies (insoluble fraction) after sonication of induced E. coli BL21 (DE3) cells. This fraction is solubilized in 6M guanidine-hydrochloride and loaded on Ni-NTA agarose (Qiagen, Chatsworth, Calif.). The column is washed with 10 volumes of 8 M urea. Refolding of PACAP receptor His-tagged N-terminal fragment is performed directly on the column using linear 6M-1 M urea gradient. The tagged protein is eluted with 0.3 M imidazole, dialyzed and used for the immunization of BALB/c mice.

The NeoI-BamHI fragment coding N-terminal extracellular part of PACAP receptor including the first putative transmembrane domain from the plasmid pNtPACAPR is recloned into the plasmid pET-28a. The resulting plasmid pET-PACAP-Nt expressed His-tagged extracellular N-terminal part of PACAP receptor containing its first transmembrane domain. (PACAPr-Nt) after the induction with 1 mM IPTG. The tagged protein is in the insoluble fraction of the cell lysate. Recombinant protein PACAPr-Nt is purified and refolded in the same way as the protein PACAPr-N expressed by the plasmid pET-PACAPr-N expressed by the plasmid pET-PACAPr-N.

Production of MAbs. Recombinant protein PACAPr-N may be used as an immunogen for Mab production. BALB/c mice are immunized subcutaneously with 50 μg of antigen emulsified in Freund's complete adjuvant. Mice are boosted on Days 20, 40, and 60 after primary immunization and tested for the presence of specific antibodies by indirect enzyme-linked immunoadsorbent assay (ELISA). Spleen cells of the best responder animal are fused with Sp2/0 myeloma cells using PEG 1500 as a fusion agent. Supernatants of viable clones are tested by indirect ELISA for the presence of antibodies to the fused protein MalPACAPr-N and further retested with recombinant protein PACAPr-Nt. On Day 14 after fusion, numerous clones growing clones are obtained and tested for expression of the recombinant PACAPr fragment. Positive hybridomas are stabilized by limiting dilution cloning using macrophage feeder layer. MAbs are produced in tissue culture supernatants and in ascitic fluids induced in Pristane-injected mice and partially purified from ascitic fluid by three-fold ammonium sulfate precipitation. Isotyping of MAbs are performed by immunodiffusion using Monoclonal Antibody Isotyping Kit (Sigma, St. Luis, Mo.).

Competitive ELISA. MAbs are conjugated with horse-radish peroxidase using periodate method. Working dilution of each conjugate is determined by direct ELISA using recombinant protein PACAPr-N. Competitive ELISA for the detection of distinct and overlapping epitopes is performed with recombinant PACAPr fragments. Ninety-six-well plates (Nunc. Denmark) are coated with 100 μL of 5 μg/mL antigen (PACAPr-N or Mal-PACAPr-N or PACAPr-Nt) in coating buffer (50 mM sodium bicarbonate buffer, pH 9.5) and incubated overnight at 4° C. The nonspecific binding is blocked with 0.5% Tween 20 in phosphate-buffered saline (PBS) for 30 min. After washing the hybridoma supernatants or purified MAbs at a concentration 10 μg/mL are added, and the plates are incubated at RT for 1 h, followed by adding peroxidase labeled MAbs at a working dilution. After incubation at RT for 1 h, the plates are washed and developed with o-phenylenediamine (OPD).

Cell lines and transient transfection of COS cells. COS cells are maintained in complete Dulbecco's modified Eagle's medium (DMEM) containing 10% fetal calf serum (FCS) and antibiotics. CHO cells are maintained in DMEM-HAMF12 medium supplemented with 10% FCS and antibiotics.

Stably transfected CHO cells expressing rat PACAPr and human neuroblastoma NB-OK cells are obtained. NB-OK cells are cultivated in RPMI-1640 medium supplemented with 10% FCS and antibiotics. Transient transfection of COS cells is performed by DEAE-dextran/chloroquine method. COS cells are transfected either with a plasmid pPACAPR (encoding either the full-length rat PACAPr) or pNtPACAPR (encoding the truncated N-terminal fragment of the receptor) or pSGS for control (mock transfection). Cells are incubated for 4.5 h with the transfection medium containing 5 μg/mL of plasmid DNA0.5 mM DEAE-dextran and 0.1 mM chloroquine in DMEM supplemented with 7% Nu-serum. After incubation, the transfection medium is carefully removed and cells are treated with 10% dimethyl sulfoxide (DMSO) in PBS for 3 min. The transfected cells are washed and incubated in complete DMEM medium at 3° C. for about 60 h prior to harvest.

Immunoblotting analysis. Cells are harvested by scraping and solubilized by boiling in 1% sodium dodecyl sulfate (SDS)/PBS. Cell lysate was factionated by SDS-PAGE in 12% minigel and electroblotted to the pilyvinyl-difluoride (PDF) membranes (Millipore, Bedford, Mass.) under semidry conditions. The membranes are blocked with 0.5% Tween 20 in PBS at RT for 30 min., washed and incubated with hybridoma supernatants for ascites fluids diluted 1:2000 at RT for 90 min. After washing, membranes are incubated with alkaline phosphatase labeled goat anti-mouse IgG (Sigma) at RT for 1 h. After several washing steps the color reaction is developed using nitroblue tetrazolium (NBT)/bromo-chlor-indolyl phosphate (BCIP) substrate.

Flow-cytometric analysis. Cells are harvested with 1 mM ethylenediaminetetraacetic acid (EDTA) in PBS, washed twice with PBS contained 0.2% bovine serum albumin (BSA) and 0.05% NaN₃ and incubated with hybridoma supernatants at 4° C. for 30 min. After two washing steps with PBS the cells are further incubated with fluorescein isothiocyanate (FITC)-labeled secondary antibodies to mouse IgG (Beckton Dickinson, Heidelberg, Germany) at 4° C. for 20 min., then washed twice with PBS, resuspended in 1% buffered formaldehyde (CelIFix, Beckton Dickinson), and analyzed using FACScan equipment (Becton Dickinson). Cell staining is compared by overlaying the histogram plots to the control samples incubated with FITC-labeled anti-mouse IgG.

Competition studies of ¹²⁵1-PACAP(1-27) with MAbs. The membranes of transfected COS cells expressing rat wildtype PACAPr are incubated with the partially purified MAbs (1/50 dilution) in binding buffer (20 mM Hepes, pH 7.4, 1 mM EGTA, 0.5% BSA, 0.5 mg/mL bacitracin, 0.04% mg/mL soybean trypsin inhibitor) at 15° C. for 2 h. Then ¹²⁵I-PACAp(1-27) (NEN, 0.1 nM) is added and incubated at 25° C. for 40 min. The ligand binding reaction was terminated by addition of 4 mL ice-cold washing buffer (20 MM HEPES, pH 7.4, 0.15 M Nail) and rapidly filtered through GO/B (Whitman, Kent, England) filters that are presoaked in 0.3% polyethylenimine for about 4 h. The filters are washed three times and dried. The radioactivity is countered in a gamma spectrometer. Non-specific binding is determined by incubation in the presence of a 1000-fold excess of unlabeled PACAP(1-27).

Human monoclonal antibodies may be made by any of the methods known in the art, such as those disclosed in U.S. Pat. No. 5,567,610, issued to Borrebaeck et al., U.S. Pat. No. 565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, new York, 1987), and Boerner et al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinant of antibody specificity (a single Fd fragment may be associated with up to ten different light chains without altering antibody specificity). Isolated Fd fragments retain the ability to specifically bind to antigen epitopes.

Within the antigen-binding region of an antibody are complementarity determining regions (CDRs) which directly interact with the epitope of the antigen, and framework regions (FRs), which maintain the tertiary structure of the paratope (see, A. G. Clark, 1986 supra; Roitt, 1991 supra). In both the heavy chain Fd fragment and the light chain of the IgG immunoglobulins, there are four framework regions (FR1-FR4) separated respectively by three complementary determining regions (CDR1-CDR3). The CDRs, and in particular the CDR3 region, and more particularly the heavy chain CDR3, are primarily responsible for antibody specificity.

The sequences of the antigen-binding Fab′ portion of the anti-PACAP receptor monoclonal antibodies identified as being useful according to the invention in the assays provided herein, as well as the relevant FR and CDR regions, can be determined using amino acid sequencing methods that are routine in the art. It is well established that non-CDR regions of a mammalian antibody may be replaced with corresponding regions of non-specific or hetero-specific antibodies while retaining the epitope specificity of the original antibody. This technique is useful for the development and use of “humanized” antibodies in which non-human CDRs are covalently joined to human FR and/or Fc/pFc′ regions to produce a functional antibody. Techniques to humanize antibodies are particularly useful when non-human animal (e.g., murine) antibodies which inhibit PACAP receptor activation are identified. These non-human animal antibodies can be humanized for use in the treatment of a human subject in the methods according to the invention. An example of a method for humanizing a murine antibody is provided in PCT International Publication No. WO 92/04381 which teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, and Fab fragments of an anti-PACAP receptor monoclonal antibody; chimeric antibodies in which the Fc and/or FR and/or CDR1 and/or CDR2 and/or light chain CDR3 regions of an anti-PACAP receptor antibody have been replaced by homologous human or non-human sequences; chimeric F(ab′)₂ fragment antibodies in which the FR and/or CDR1 and/or CDR2 and/or light chain CDR3 regions of an anti-PACAP receptor antibody have been replaced by homologous human or non-human sequences; and chimeric Fab fragment antibodies in which the FR and/or CDR1 and/or CDR2 and/or light chain CDR3 regions have been replaced by homologous human or non-human sequences. Thus, those skilled in the art may alter an anti-PACAP receptor antibody by the construction of CDR grafted or chimeric antibodies or antibody fragments containing, all or part thereof, of the disclosed heavy and light chain V-region CDR AA sequences (Jones et al., Nature 321:522, 1986; Verhoeyen et al., Science 39:1534, 1988 and Tempest et al., Bio/Technology 9:266, 1991), without destroying the specificity of the antibodies for PACAP receptor. Such CDR grafted or chimeric antibodies or antibody fragments can be effective in inhibiting PACAP receptor activation in animals (e.g. cattle) and humans.

In preferred embodiments, the chimeric antibodies of the invention are fully human monoclonal antibodies. As noted above, such chimeric antibodies may be produced in which some or all of the FR regions of PACAP receptor have been replaced by other homologous human FR regions. In addition, the Fc portions may be replaced so as to produce IgA or IgM as well as IgG antibodies bearing some or all of the CDRs of the Anti-PACAP receptor antibody. Of particular importance is the inclusion of the anti-PACAP receptor heavy chain CDR3 region and, to a lesser extent, the other CDRs of anti-PACAP receptor. Such fully human chimeric antibodies will have particular utility in that they will not evoke an immune response against the antibody itself.

It is also possible, in accordance with the present invention, to produce chimeric antibodies including non-human sequences. Thus, one may use, for example, murine, ovine, equine, bovine or other mammalian Fc or FR sequences to replace some or all of the Fc or FR regions of the anti-PACAP receptor antibody. Some of the CDRs may be replaced as well. Again, however, it is preferred that at least the heavy chain CDR3 region of the anti-PACAP receptor antibody be included in such chimeric antibodies and, to a lesser extent, it is also preferred that some or all of the other CDRs of anti-PACAP receptor be included. Such chimeric antibodies bearing non-human immunoglobulin sequences admixed with the CDRs of the human anti-PACAP receptor monoclonal antibody are not preferred for use in humans and are particularly not preferred for extended use because they may evoke an immune response against the non-human sequences. They may, of course, be used for brief periods or in immunosuppressed individuals but, again, fully human antibodies are preferred. Because, however, such antibodies may be used for brief periods or in immunosuppressed subjects, chimeric antibodies bearing non-human mammalian Fc and FR sequences but including at least the anti-PACAP receptor heavy chain CDR3 are contemplated as alternative embodiments of the present invention.

For therapeutic or prophylactic uses, the antibodies of the present invention are preferably intact antibody molecules including the Fc region. Such intact antibodies will have longer half-lives than smaller fragment antibodies (e.g. Fab) and are more suitable for intravenous, intraperitoneal, intramuscular, intracavity, subcutaneous, or transdermal administration.

Fab fragments, including chimeric Fab fragments, may be preferred in methods in which the antibodies of the invention are administered directly to a local tissue environment. For example, the Fab fragments are preferred when the antibody of the invention is administered directly to the site of bladder tissue. Fabs offer several advantages over F(ab′)₂ and whole immunoglobulin molecules for this therapeutic modality. First, because Fabs have only one binding site for their cognate antigen, the formation of immune complexes is precluded whereas such complexes can be generated when bivalent F(ab′)₂ s and whole immunoglobulin molecules encounter their target antigen. This is of some importance because immune complex deposition in tissues can produce adverse inflammatory reactions. Second, because Fabs lack an Fc region they cannot trigger adverse inflammatory reactions that are activated by Fc, such as activation of the complement cascade. Third, the tissue penetration of the small Fab molecule is likely to be much better than that of the larger whole antibody. Fourth, Fabs can be produced easily and inexpensively in bacteria, such as E. coli, whereas whole immunoglobulin antibody molecules require mammalian cells for their production in useful amounts. Production of Fabs in E. coli makes it possible to produce these antibody fragments in large fermenters which are less expensive than cell culture-derived products.

Smaller antibody fragments and small binding peptides having binding specificity for the PACAP receptor which can be used to inhibit PACAP receptor activation also are embraced within the present invention. For example, single-chain antibodies can be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated VH single domain, also have been reported (see, for example, Ward et al., Nature 341:644-646 (1989)).

Small peptides also may easily be synthesized or produced by recombinant means by those of skill in the art. Using routine procedures known to those of ordinary skill in the art, one can determine whether a peptide which binds to PACAP receptor is useful according to the invention by determining whether the peptide is one which inhibits the activation of PACAP receptor in a PACAP receptor activation assay. Several routine assays may be used to easily identify such peptides. Screening assays for identifying peptides of the invention are performed for example, using phage display procedures such as those described in Hart, et al., J. Biol. Chem. 269:12468 (1994). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to PACAP receptor are obtained by selecting those phages which express on their surface a ligand that binds to the PACAP receptor. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding to the PACAP receptor. Alternatively, such peptide ligands can be selected from combinatorial libraries of peptides containing one or more amino acids. Such libraries can further be synthesized which contain non-peptide synthetic moieties which are less subject to enzymatic degradation compared to their naturally-occurring counterparts.

In addition to the above-described binding assays the following may also be employed to screen molecules useful according to the invention. Once a molecule useful according to the invention is identified it is possible to produce anti-idiotypic antibodies which can be used to screen other monoclonal antibodies to identify whether the antibody has the same binding specificity as an antibody of the invention. Such anti-idiotypic antibodies can be produced using well-known hybridoma techniques (Kohler and Milstein, Nature, 256:495, 1975). An anti-idiotypic antibody is an antibody which recognizes unique determinants present on the putative inhibitor. These determinants are located in the hypervariable region of the antibody. It is this region which binds to a given epitope and, thus, is responsible for the specificity of the antibody. An anti-idiotypic antibody can be prepared by immunizing an animal with the monoclonal antibody which was identified (screening antibody). The immunized animal will recognize and respond to the idiotypic determinants of the screening antibody and produce an antibody to these idiotypic determinants. By using the anti-idiotypic antibodies of the immunized animal, which are specific for the screening antibody, it is possible to identify other clones with the same idiotype as the screening antibody. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the specificity for an epitope of PACAP receptor which inhibits the activation of the PACAP receptor.

The invention also includes the use of a “dominant negative PACAP receptor” polypeptide. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor PACAP binds a ligand but does not transmit a signal in response to binding of the ligand can reduce the biological effect of expression of the ligand. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide as used herein in a cell is a reduction in function of active PACAP receptor. One of ordinary skill in the art can assess the potential for a dominant negative variant of a protein, and using standard mutagenesis techniques to create one or more dominant negative variant polypeptides. For example, one of ordinary skill in the art can modify the sequence of the PACAP receptor by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for the ability to prevent PACAP receptor activation. Other similar methods for creating and testing dominant negative variants of a protein will be apparent to one of ordinary skill in the art.

In general, the inhibitors of PACAP receptor activity of the invention are useful for treating disorders characterized by the increased expression of PACAP. In particular the inhibitors of the invention are useful for treating disorders characterized by the increased expression of PACAP in the micturition (urinary) reflex pathways. Disorders such as urinary bladder inflammation and chronic cystitis increase PACAP immunoreactivity in micturition pathways (Vizzard, J. Comp. Neuol. 420:335-348 (2000)).

The inhibitors of PACAP receptor activity of the invention are useful for treating overactive bladder and pelvic floor pain syndrome in a subject. The term “treatment” and “treating” is intended to encompass also prophylaxis, therapy and cure. The terms “treatment” and “treating” as used herein refer to inhibiting completely or partially the expression or activity of PACAP receptor, as well as inhibiting an increase in the expression or activity of PACAP receptor.

The terms “reduce” or “eliminate” refers to reducing or eliminating the symptoms of the disorders as determined by cystometric parameters such as filling pressure (pressure at the beginning of the bladder filling), threshold pressure (bladder pressure immediately prior to micturition), micturition pressure (the maximal bladder pressure during micturition), micturition interval (time between micturition events), void volume and presence or absence of non-voiding bladder contractions (NVC). In addition the reduction and elimination of the disorders can be determined from the observation of clinical symptoms. These factors are well known to those of ordinary skill in the art and can be addressed with no more than routine experimentation and examination.

“Pelvic floor pain syndrome” is a disorder characterized by chronic prostatitis, chronic genitourinary pain (perineal, suprapubic, penile, ejaculatory, etc.), variable obstructive and irritative voiding symptoms and sexual dysfunction, without a history of recurrent urinary tract infection and/or demonstration of uropathogenic bacteria localized to the prostate gland. Pelvic floor pain syndrome is associated with one or more of the following symptoms: genitourinary pain, irritative and obstructive voiding symptoms, deep perineal pain, pain in the penis, testicles, inguinal area, suprapubic area, back and on ejaculation; urgency, frequency, nocturia, hesitancy, slow stream and poor bladder emptying and dysuria; severe anal sphincter spasms, pelvic floor tenderness; may be associated with infection and inflammation.

The pelvic floor pain syndrome may be associated with a condition such as: overactive bladder, interstitial cystitis, outlet obstruction, benign prostatic hyperplasia, detrusor smooth muscle, bladder afferents, neurogenic bladder, bacterial and non-bacterial infections, incontinence.

Pelvic floor pain syndrome may also be known as or associated with: painful bladder syndrome, painful bladder disease complex, nonbacterial cystitis, chronic cystitis, pelvic pain, daytime urinary frequency, nighttime urinary frequency, urinary urgency, interstitial cystitis, urinary tract infection, urethral diverticulum, Bartholin gland infection, Skene gland infection, vulvodynia, vulvovestibulitis, tuberculous cystitis, eosinophilic cystitis, vaginitis, schistosomiasis, pelvic malignancy, pelvic mass, fibroid, endometrioma, endometriosis, mittelschmerz, pelvic inflammatory disease, genital atrophy, bladder cancer, carcinoma in situ, radiation cystitis, overflow incontinence, acontractile detrusor, prostatodynia, chronic pelvic pain syndrome, bladder outlet obstruction, open bladder neck, intrinsic sphincteric deficiency, urolithiasis, urethritis, detrusor hyperreflexia, Parkinson disease, lumbosacral disk disease, spinal stenosis, spinal tumor, multiple sclerosis, cerebrovascular accident, dysfunctional voiding, pelvic floor myalgia, degenerative joint disease, hernia, inflammatory bowel disease, gastrointestinal neoplasm, diverticulitis. The term “interstitial cystitis” refers to a clinical syndrome characterized by daytime and nighttime urinary frequency, urgency, and pelvic pain of unknown etiology.

The inhibitors of PACAP activity are also useful for treating overactive bladder disorder in a subject. The term “overactive bladder” is used to describe a bladder that contracts more often than it should, so that a person feels the need to urinate more frequently and/or urgently than necessary. An overactive bladder usually, but not always, causes urinary incontinence. Overactive bladder disorder is also known as or associated with: bladder hyperflexia, interstitial cystitis, outlet obstruction, benign prostatic hyperplasia, detrusor smooth muscle, bladder afferents, neurogenic bladder, bacterial and non-bacterial infections, and incontinence.

“Urinary incontinence” is the inability to control urinary functions effectively; the result is that a person is unable to make it to the toilet in time. Incontinence is often temporary, and it almost always results from an underlying medical condition. It is a condition that ranges from mild leakage to uncontrollable wetting. The most common types of urinary incontinence are:

Stress incontinence happens when urine leaks during exercise, coughing, sneezing, laughing, lifting heavy objects, or other body movements that put pressure on the bladder. It is the most common type of incontinence and can almost always be cured.

Urge incontinence occurs when an overactive bladder contracts without your wanting it to do so. Although healthy people can have urge incontinence, it is often found in people who have diabetes, stroke, dementia, Parkinson's disease, or multiple sclerosis. It can also be a warning sign of early bladder cancer. In men, it is often a sign of an enlarged prostate.

Overflow incontinence happens when small amounts of urine leak from a bladder that is always full. In older men, this can occur when the flow of urine from the bladder is blocked, usually by an enlarged prostate. It can sometimes be prevented by medication when early symptoms of prostate enlargement, such as frequent urination, appear. Some people with diabetes also have overflow incontinence.

Functional incontinence happens in many older people who have relatively normal urine control but who have a hard time getting to the toilet in time because of arthritis or other crippling disorders.

“Benign prostatic hyperplasia” is a common, noncancerous condition of men above 50 years old (it affects about 30 to 50% of older men). The enlarged prostate (a walnut-sized gland at the base of the penis) obstructs the urethra (the small tube that carries urine from the bladder) and causes frequent urination.

The term “neurogenic bladder” refers to loss of normal bladder function due to damage of the nervous system. For experimental purposes a neurogenic bladder can be studied by inducing a chronic spinal cord injury in laboratory animals. For example, a complete spinal cord transaction is performed in adult, female rats anesthetized with isoflurane (2-3%). Animals are evaluated using conscious cystometry six-weeks following surgery when each animal has recovered automatic micturition.

The inhibitors of PACAP receptor activity of the invention are also useful for modulating micturition frequency in a subject. The term “micturition” means urinary voiding or urination.

The agents of the invention are administered in “effective amounts”. The “effective amount” is that amount which is capable of at least partially preventing, reversing, reducing, decreasing, ameliorating, or otherwise suppressing the particular disorder being treated. A therapeutically effective amount can be determined on an individual basis and will be based, at least in part, on consideration of the species of mammal, the mammal's age, sex, size, and health; the inhibitor of PACAP receptor activity used, the type of delivery system used; the time of administration relative to the severity of the disease; and whether a single, multiple, or controlled-release dose regiment is employed. In general, an effective amount for treating an overactive bladder disorder or pelvic pain syndrome will be that amount necessary to inhibit the onset or progression of, or to reduce or eliminate the overactive bladder or pelvic floor pain syndrome. This can be determined by observing symptoms of pelvic floor pain syndrome as well as the cystometric parameters described above. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

A variety of administration routes are available. The particular mode selected will depend of course, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. The inhibitors of PACAP receptor activity of the invention can be administered by any method which allows the inhibitor to reach the target cells, e.g., PACAP receptor presenting cells. These methods include, e.g., injection, infusion, deposition, implantation, anal or vaginal supposition, oral ingestion, inhalation, topical administration, administration by catheter or any other method of administration where access to the target cell by the inhibitor is obtained. The term “parenteral” includes intravenous, intradermal, subcutaneous, intramuscular, or intraperitoneal injections. In some embodiments, the injections can be given at multiple locations.

The compositions may conveniently be presented in unit dosage form and may be prepared by any of the methods well known in the art of pharmacy. All methods include the step of bringing the conjugates of the invention into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Compositions suitable for oral administration may be presented as discrete units such as capsules, cachets, tablets, or lozenges, each containing a predetermined amount of the active compound. Other compositions include suspensions in aqueous liquors or non-aqueous liquid such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

The active agents thus can be provided in pharmaceutical preparations. When administered, the pharmaceutical preparations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrocholoric, hydrobromic, sulphuric, nitric, phosphoric, maleic, acetic, salicyclic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts. Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The pharmaceutical preparations of the present invention contain an effective amount of an agent included with a pharmaceutically-acceptable carrier. The term “pharmaceutically-acceptable carrier” as used herein means one or more compatible solid or liquid filler, dilutants or encapsulating substances which are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy. Carrier formulations suitable for oral, topical, subcutaneous, intravenous, intramuscular, etc. can be found in Remington's Pharmaceutical Sciences, Mack Publishing Company, Easton, Pa., USA.

The compounds useful in the invention may be used alone, without other active agents. They also may be used together with other active agents, such as anti-inflammatory agents, anti-bacterial agents, or agents known useful in treating the conditions described herein. The agents may be delivered separately from one another or in the form of a cocktail of two or more agents. A cocktail is a mixture of any one of the compounds useful with this invention with another active agent.

The inhibitors of PACAP receptor activity can be delivered alone or in association with a vector. In its broadest sense, a “vector” is any vehicle capable of facilitating the delivery of an inhibitor of PACAP receptor activity to a PACAP receptor. Preferably, the vectors transport the inhibitor of PACAP receptor activity to the PACAP receptor with reduced degradation relative to the extent of degradation that would result in the absence of the vector. Vectors of the invention could also help to reduce degradation of the inhibitor of PACAP receptor activity in the lysosomal environment. The pH of the lysosome is very low and can interfere with the normal activity of compounds. Preferably, the inhibitor of PACAP receptor activity are delivered to the lysosome in a form which is resistant to the low pH of the lysosome. Optionally, a “targeting ligand” can be attached to the vector to selectively deliver the vector to a cell which expresses on its surface the PACAP receptor. In general, the vectors useful in the invention are divided into two classes: biological vectors and chemical/physical vectors. Biological vectors and chemical/physical vectors are useful for delivery of inhibitor of PACAP receptor activity to a target cell.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be attached to the nucleic acid sequences of the invention. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other vectors not named but known in the art.

Preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Another preferred virus for certain applications is the adeno-associated virus, a double-stranded DNA virus. The adeno-associated virus can be engineered to be replication -deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In addition to the biological vectors, chemical/physical vectors may be used to deliver a inhibitor of PACAP receptor activity to a target PACAP receptor. As used herein, a “chemical/physical vector” refers to a natural or synthetic molecule, other than those derived from bacteriological or viral sources. A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77).

Liposomes may be targeted to a particular tissue, by coupling the liposome to a specific ligand such as a monoclonal antibody, sugar, glycolipid, or protein. Such ligands may easily be identified by binding assays well known to those of skill in the art. Additionally, the vector may be coupled to a nuclear targeting peptide, which will direct the vector to the nucleus of the host cell.

Liposomes are commercially available from Gibco BRL, for example, as LIPOFECTIN and LIPOFECTACE, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one particular embodiment, the preferred vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO 95/24929, entitled “Polymeric Gene Delivery System”. PCT/US/0307 describes a biocompatible, preferably biodegradable polymeric matrix for containing an exogenous gene under the control of an appropriate promotor. The polymeric matrix is used to achieve sustained release of the exogenous gene in the patient.

The polymeric matrix preferably is in the form of a microparticle such as a microsphere (wherein the PACAP receptor or inhibitor is dispersed throughout a solid polymeric matrix) or a microcapsule (wherein the PACAP receptor inhibitor is stored in the core of a polymeric shell). Other forms of the polymeric matrix for containing the inhibitor of PACAP receptor activity include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the inhibitor of PACAP receptor activity are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the chemical/physical vector is a biocompatible microsphere that is suitable for oral delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the PACAP receptor and inhibitors to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector when the inhibitor is a nucleic acid. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of an inhibitor of PACAP receptor activity into a target cell include calcium phosphate and other chemical mediators of intracellular transport, microinjection compositions, electroporation and homologous recombination compositions (e.g., for integrating a nucleic acid into a pre-selected location within the target cell chromosome).

Administration by catheter implant is an important mode of administration of the invention, such as by intravesical and by intrathecal catheter implants. The term “intravesical” means within the bladder, and the term “intrathecal” refers to administration in the fluid filled space between the thin layers of tissue that cover the spinal cord. Intravesical and intrathecal catheter placement may be performed by one of ordinary skill in the art using well known procedures.

Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the inhibitor of PACAP receptor activity with an aerosol in an inhaler, either alone or attached to a carrier that can be absorbed. For systemic administration, it may be preferred that the inhibitor of PACAP receptor activity is encapsulated in liposomes.

In certain embodiments of the invention, the administration can be designed so as to result in sequential exposure of the inhibitor of PACAP receptor activity over some period of time, e.g., hours, days, weeks, months or years. This can be accomplished by repeated administrations of the inhibitor of PACAP receptor activity, by one of the methods described above, or alternatively, by a controlled-release delivery system in which the inhibitor of PACAP receptor activity is delivered to the mammal for a prolonged period without repeated administrations. By controlled-release delivery system, it is meant that total release of the inhibitor does not occur immediately upon administration, but rather is delayed for some period of time. Release can occur in bursts or it can occur gradually and continuously. Administration of such a system can be, e.g., by long-lasting oral dosage forms, bolus injections, transdermal patches, and subcutaneous implants.

Examples of systems in which release occurs in bursts includes, e.g., systems in which the inhibitor is entrapped in liposomes which are encapsulated in a polymer matrix, the liposomes being sensitive to specific stimuli, e.g., temperature, pH, light or a degrading enzyme and systems in which the inhibitor is encapsulated by an ionically-coated microcapsule with a microcapsule core degrading enzyme. Examples of systems in which release of the inhibitor is gradual and continuous include, e.g., erosional systems in which the inhibitor is contained in a form within a matrix and effusional systems in which the inhibitor permeates at a controlled rate, e.g., through a polymer. Such sustained release systems can be e.g., in the form of pellets, or capsules.

The inhibitor of PACAP receptor activity can be suspended in a liquid, e.g., in dissolved form or colloidal form. The liquid can be a solvent, partial solvent, or non-solvent. In many cases, water or an organic liquid can be used.

In some embodiments, the concentration of the inhibitor if administered systemically is at a dose of about 1.0 mg to about 2000 mg for an adult of 70 kg body weight, per day. In other embodiments, the dose is about 10 mg to about 1000 mg/70 kg/day. In yet other embodiments, the dose is about 100 mg to about 500 mg/70 kg/day. Preferably, the concentration of the inhibitor, if applied topically, is about 0.1 mg to about 500 mg/gm of ointment or other base, more preferably about 1.0 mg to about 100 mg/gm of base, and most preferably, about 30 mg to about 70 mg/gm of base. The specific concentration partially depends upon the particular inhibitor used, as some are more effective than others. The dosage concentration of the inhibitor actually administered is dependent at least in part upon the particular disorder being treated, the final concentration of inhibitor that is desired at the site of action, the method of administration, the efficacy of the particular inhibitor, the longevity of the particular inhibitor, and the timing of administration relative to the severity of the disease. Preferably, the dosage form is such that it does not substantially deleteriously effect the mammal. The dosage can be determined by one of ordinary skill in the art employing such factors and using no more than routine experimentation.

A “subject” shall mean a human or non-human mammal, including but not limited to, a dog, cat, horse, cow, pig, sheep, goat, chicken, primate, rat, and mouse.

The materials for use in the invention, such as the inhibitors of PACAP receptor activity are ideally suited for the preparation of a kit. Such a kit may comprise a carrier means being compartmentalized to receive in close confinement one or more container means such as vials, tubes, and the like, each of the container means comprising one of the separate elements to be used in the method. For example, one of the container means may comprise a peptide or a monoclonal antibody of the invention which selectively binds for use as a positive control in the assay. The kit may also have containers comprising inhibitors of PACAP receptor activity and a substrate for detecting activation of the PACAP receptor. Additionally the kit may include containers for buffer(s) useful in the assay. In other examples, the kit may comprise packaging material and a formulation comprising an inhibitor of PACAP receptor activity, wherein the packaging material comprises a label or package insert that states that the formulation can be administered to a subject having pelvic floor syndrome in an amount, at a frequency, and for a duration effective to reduce or eliminate the pelvic floor syndrome.

In another example of the invention, the kit may comprise packaging material and a formulation that comprises an inhibitor of PACAP receptor activity, wherein the packaging material comprises a label or package insert that states that the formulation can be administered to a subject having overactive bladder disorder in an amount, at a frequency, and for a duration effective to reduce or eliminate the overactive bladder.

In yet another example of the invention, the kit comprises packaging material and a formulation that comprises an inhibitor of PACAP receptor activity, wherein the packaging material comprises a label or package insert that states that the formulation can be administered to a subject in need of modulating micturition frequency in an amount, at a frequency, and for a duration effective to decrease the micturition frequency.

### EXAMPLES

### Materials and Methods

### Experimental Procedures for Examples 1-5

Cyclophosphamide (CYP)-Induced Cystitis-Acute, Intermediate or Chronic

Chemical cystitis was induced in adult female Wistar rats by cyclophosphamide (CYP), which is metabolized to acrolein, an irritant eliminated in the urine. CYP (Sigma ImmunoChemicals, St. Louis, Mo.) was administered in one of the following ways: (1) 4 hr (150 mg/kg; i.p.) prior to euthanasia of the animals to elicit acute inflammation (n=6); (2) 48 hr (150 mg/kg; i.p.) prior to euthanasia to examine an intermediate inflammation (n=6) or (3) administered every third day for 10 days to elicit chronic inflammation (n=8; 75 mg/kg; i.p). All injections of CYP were performed under isoflurane (2%) anesthesia.

Chronic Spinal Cord Injury (SCI)

A complete spinal cord transaction was performed in adult, female rats anesthetized with isoflurane (2-3%). Animals received antibiotic and analgesic treatment following surgery. Animals were evaluated using conscious cystometry six-weeks following surgery when each animal had recovered automatic micturition. The University of Vermont IACUC approved all experimental procedures (protocol #02-108, 03-030, 03-148D) involving animal use. Animal care was under the supervision of the University of Vermont's Office of Animal Care in accordance with the Association for Assessment and Accreditation of Laboratory Animal Care and National Institutes of Health guidelines. All efforts were made to minimize animal stress/distress and suffering and to use the minimum number of animals. Currently, no alternatives exist to the use of whole, live animals in the present study.

Control Experiments

Control animals (n=12) received a corresponding volume of saline (0.9%; i.p.) under isoflurane (2%) anesthesia for cystitis studies injected. Spinal intact rats served as controls for the spinal transaction studies.

Intrathecal Catheter Placement

Rats were anesthetized with isoflurane anesthesia (2-3%) and the atlanto-occiptal membrane was exposed by aseptic surgical technique. Polyethylene tubing (PE-10, Clay Adams, Parsippany, N.J.) was inserted under the membrane and passed to the lumbosacral spinal cord (L6-S1). Correct catheter placement was confirmed at time of euthanasia. The intrathecal catheter was secured in place with a small drop of dental cement and the incision was closed in two layers. The intravesical catheter was then inserted.

Intravesical Catheter Placement

A lower midline abdominal incision was performed under general anesthesia (ketamine 60 mg/kg, xylazine 10 mg/kg, i.p.), and polyethylene tubing (PE-50) with the end flared by heat was inserted into the dome of the bladder and secured in place with a 6-0 nylon purse string suture. The distal end of the tubing was sealed, tunneled subcutaneously and externalized at the back of the neck, out of the animal's reach. Abdominal and neck incisions were closed with 4-0 nylon sutures. Animals were maintained for 72 hours after survival surgery to ensure complete recovery.

Cystometry

Control rats and rats treated with CYP (acute; 4 hr or intermediate; 48 hr) or SCI rats and also treated with a specific PACAP antagonist, PACAP6-38 or vehicle (0.9% saline), were evaluated with cystometry. For conscious cystometry, a rat was placed unrestrained in a cage. Prior to the start of the recording, the bladder was emptied and the catheter was connected via a T-tube to a pressure transducer (Grass® Model PT300, West Warwick, R.I.) and microinjection pump (Harvard Apparatus 22, South Natick, Mass.). We infused a 0.9% saline solution at room temperature into the bladder at a rate of 10 ml/hour. Intravesical pressure was recorded continuously using a Neurodata Acquisition System (Grass® Model 15, Astro-Med, Inc, West Warwick, R.I.). At least four reproducible micturition cycles were recorded after the initial stabilization period of 25-30 minutes. The following cystometric parameters were recorded in each animal: filling pressure (pressure at the beginning of the bladder filling), threshold pressure (bladder pressure immediately prior to micturition), micturition pressure (the maximal bladder pressure during micturition), micturition interval (time between micturition events), void volume and presence or absence of non-voiding bladder contractions (NVC). For the present study, NVCs were defined as increases in bladder pressure of at least 7 cm H₂O without release of urine.

Drug Treatment

The effects of a specific PAC₁ receptor antagonist, PACAP6-38 (i.t. or intravesical) (Peninsula Pharmaceuticals) or vehicle (0.9% saline, i.t. or intravesical; artificial cerebrospinal fluid, aCSF, i.t.) on lower urinary tract function in rats treated with CYP (4 hr, 48 hr or chronic cystitis) were evaluated. Separate groups of rats were used to evaluate the effects of PACAP6-38 given either through the i.t. catheter or the intravesical catheter. After recording at least three reproducible micturition cycles, PACAP6-38 or saline was administered through the catheters (i.t. or intravesical) and the effects evaluated.

Statistics

All values are means±S.E.M. Comparisons of urodynamic parameters between control and experimental treatments were made using the Fischer exact test. When F ratios exceeded the critical value (P≦0.05), the Dunnett's post-hoc test was used to compare the control mean with each experimental mean.

### Experimental Procedures for Examples 6-13

Experimental Animals

Adult female Wistar rats (Charles River, Canada; 150-200 g; spinal cord intact (control; n=6); spinal cord injury (SCI; 48 hours, 1 week, 2 weeks, 6 weeks after SCI; n=6 for each) were used for anatomical studies. For studies examining PACAP immunostaining in the urinary bladder, spinal cord intact rats and those with SCI 5 days or 3 weeks prior to injury were used.

Spinal Cord Transection

Spinal cord transection was performed under isoflurane anesthesia (2-2.5%) 48 hr to 6 weeks prior to intracardiac perfusion as previously described (Qiao and Vizzard, 2002b; Vizzard, 1997; Vizzard, 1999; Vizzard, 2000c). Briefly, the dorsal T7-T9 vertebrae were removed and the spinal cord was completely transected. The space between the retracted ends of the spinal cord was packed with Gelfoam (Upjohn Company of Canada, Ontario) and the incision sutured. Complete spinal transection was visually confirmed at the time of euthanasia and tissue dissection (see below). Following surgery, the animals were housed in Alpha-Dri (Shepherd Specialty Papers, Kalamazoo, Mich.) lined cages and their bladders were manually expressed two to three times a day. An antibiotic (150 mg/kg ampicillin,s.c.) was administered prophylactically 1 day prior to surgery and for 3 days postoperatively. The analgesic buprenorphine (0.01 mg/kg, s.c.) was delivered postoperatively every twelve hours for a total of four doses. Studies were restricted to female animals because manual expression of the bladder is more easily accomplished in the female rat because of the shorter and concomitantly smaller region occupied by the external urethral sphincter compared to male rats. Animals were studied 48 hr to 6 weeks after spinal transection. All animals studied at 6 weeks after SCI had developed bladder-to-bladder and perineal-to-bladder reflexes and 5 out of 6 animals had developed these reflexes 2 weeks after SCI.

Retrograde Labeling of Bladder Afferent Neurons

Five to seven days prior to perfusion, Fast Blue (FB; 4%, weight/volume; Polyol, Gross Umstadt, Germany) was injected into the bladder to retrogradely label bladder afferent neurons. As previously described (Qiao and Vizzard, 2002a; Qiao and Vizzard, 2002b; Vizzard, 2000e) a total volume of 40 μl divided into six to eight injections was injected into the dorsal surface of the bladder wall with particular care to avoid injections into the bladder lumen, major blood vessels, or overlying fascial layers. At each injection site, the needle was kept in place for several seconds after injection, and the site was washed with saline to minimize contamination of adjacent organs with FB. For rats surviving 48 hours after SCI, FB was injected prior to spinal transection. For rats surviving 1 week after SCI, FB was injected at the time of the spinal transection procedure.

Tissue Processing

After spinalization (range 48 hr to 6 weeks), animals were deeply anesthetized with isoflurane (3-4%) and then euthanized via intracardiac perfusion first with oxygenated Krebs buffer (95% O2, 5% CO₂) followed by 4% paraformaldehyde. After perfusion, the spinal cord and DRG were quickly removed and postfixed for 6 hours. Tissue was then rinsed in phosphate buffered saline (PBS; 0.1 M NaCl, in phosphate buffer, pH 7.4) and placed in ascending concentrations of sucrose (10-30%) in 0.1 M PBS for cryoprotection. Spinal cord segments were identified based upon at least two criteria: (1) the T13 DRG exists after the last rib and (2) the L6 vertebra is the last moveable vertebra followed by the fused sacral vertebrae. Another less precise criterion is the observation that the L6 DRG are the smallest ganglia following the largest, L5 DRG. DRG sections from the (L1, L2, L4-S1) spinal cord segments were sectioned parasagitally at a thickness of 20 μm on a freezing microtome. Some DRG (L1, L2, L6, S1) were specifically chosen for analysis based upon the previously determined segmental representation of urinary bladder circuitry (de Groat et al., 1994; Donovan et al., 1983; Keast and de Groat, 1992; Nadelhaft and Vera, 1995). Bladder afferents are not distributed within the L4-L5 DRG (Donovan et al., 1983; Keast and de Groat, 1992) that contain only somatic afferents nor are neurons that are involved in urinary bladder function observed in the L4-L5 spinal segments (de Groat et al., 1994; Nadelhaft and Vera, 1995). Thus, the L4-L5 DRG served as internal controls for these studies. Tissues from control animals with an intact spinal cord were handled in an identical manner to that described above.

Whole Mount Bladder Preparation

Animals, with SCI performed 5 days or 3 weeks prior, were euthanized as described above. Prior to intracardiac perfusion with fixative, the urinary bladder was dissected and placed into Krebs solution (119.0 mmol NaCl, 4.7 mmol KCl, 24.0 mmol NaHCO₃, 1.2 mmol KH₂PO₄, 1.2 mmol MgSO₄.7H₂O, 11.0 mmol glucose, 2.5 mmol CaCl2) (Zvarova et al., 2004). The bladder was cut open through the urethra in the midline and pinned flat on a sylgard-coated dish. After maximal stretch of the tissue, the bladder was incubated for 1.5 hr at room temperature in cold fixative (2% paraformaldehyde+0.2% picric acid) and the urothelium was removed. Urothelium and detrusor smooth muscle were examined separately for PACAP immunoreactivity (IR) by a free-floating technique. In some preparations, detrusor smooth muscle and urothelium stained for PACAP-IR (see below), was also immunostained for the capsaicin receptor (vanilloid receptor 1, VR1; 1:1000; Chemicon International, Temecula, Calif.).

Pituitary Adenylate Cyclase Activating Polypeptide (PA CAP) Immunohistochemistry

Spinal cord sections, detrusor and urothelium for both control and SCI animals were processed for PACAP-IR by using a free-floating method. PACAP staining in the spinal cord was evaluated in control (spinal intact) and SCI (6 weeks) rats when all SCI rats had developed spinal micturition reflexes. DRG were immunostained using an on-slide processing technique. Groups of control animals and experimental animals were processed simultaneously to decrease the incidence of variation in staining and background that can occur between sections and between animals. Spinal cord sections, detrusor and urothelium were incubated overnight at room temperature with PACAP antisera (mouse monoclonal antibody (code MabJH6F10); 1:20; diluted in potassium PBS (0.01 M KPBS) with Triton X-100 (0.04%). Additional tissue sections were incubated 48 hours at 4° C. with commercially available PACAP-27 or PACAP-38 antisera (rabbit anti-PACAP-27 or -38; 1:3000; Phoenix Pharmaceuticals, Inc., CA). Tissue was rinsed in 0.1 M PBS (3 rinses×10 min each). PACAP-immunoreactivity (PACAP-IR) in DRG (20 μm) was detected using an on-slide immunofluorescence technique. DRG sections were incubated for 48 hours in a humidified box at 4° C. with PACAP antisera (monoclonal antibody; code MabJH6F10); 1:10 diluted in KPBS plus 0.4% Triton X-100. Additional tissue sections were incubated 48 hours at 4° C. with commercially available PACAP-27 or PACAP-38 antisera (rabbit anti-PACAP-27 or -38; 1:2000; Phoenix Pharmaceuticals, Inc., Mountain View, Calif.). Tissue was then incubated with Cy3-conjugated donkey anti-mouse IgG (1:400; Jackson ImmunoResearch Laboratories, West Grove, Pa.) or Cy3-conjugated goat anti-rabbit IgG (1:500; Jackson ImmunoResearch Laboratories, West Grove, Pa.) for two hours at room temperature. Tissue was rinsed in PBS (3 rinses×10 min each), mounted on gelatin-coated slides and coverslipped with Citifluor (Citifluor Ltd., London, UK). Control sections in which primary antibody or secondary antibody was replaced with diluent (KPBS plus 0.4% Triton X-100) or with antibody preabsorbed with PACAP (20 μg/ml) diluent were negative. The specification of the monoclonal antibody for PACAP has been characterized previously (Hannibal et al., 1995). Commercially available PACAP-27 and PACAP-38 antisera (Phoenix Pharmaceuticals, Inc., Calif.) do not exhibit cross-reactivity with vasoactive intestinal polypeptide as indicated by the manufacturer.

Data Analysis

Tissues were examined under an Olympus fluorescence photomicroscope for visualization of Cy3 and FB. Cy3 was visualized with a filter with an excitation range of 560-596 nm and an emission range from 610-655 nm. In DRG from spinal intact and SCI (range 48 hr-6 weeks SCI) rats, PACAP-IR cell profiles were counted in 10 to 15 sections of each selected DRG (L1, L2, L4-S1). Only cell profiles with a nucleus were quantified. DRG sections with FB-labeled cells were viewed with a filter with an excitation wavelength 340-380 nm and an emission wavelength of 420 nm. Cells colabeled with FB+PACAP-IR were similarly counted. Numbers of PACAP-IR cell profiles per DRG section are presented (mean±S.E.M.). The percentage of presumptive bladder afferent cells (FB-labeled) expressing PACAP-IR in each DRG examined is also presented (mean±S.E.M). The results are not corrected for double counting. Comparisons between spinal intact and SCI groups were made using analysis of variance. Percentage data were arcsin transformed to meet the requirements of this statistical test. Animals, processed and analyzed on the same day, were tested as a block in the analysis of variance. Thus, day was treated as a blocking effect in the model. Two variables were being tested in the analysis: (1) experimental manipulation versus control situation and (2) the effect of day (i.e., tissue from groups (experimental and control) of animals were processed on different days). When F ratios exceeded the critical value (p_{—}0.05), the Dunnett's test was used to compare each experimental mean to the control mean.

Assessment of Positively Stained DRG Cells

Staining observed in experimental tissue was compared to that observed from experiment matched negative controls. DRG cells exhibiting immunoreactivity that was greater than the background level observed in experiment-matched negative controls were considered positively stained. Positively counted cells were not further divided into categories of different staining intensities.

Spinal Cord Densitometry

The density of PACAP-IR in specific regions of spinal cord in control rats and those with a 6 week SCI was determined with densitometry analysis (Image-Pro express, version 4.0, Media Cybernetics, L.P.) as previously described (Vizzard, 1999; Vizzard, 2000e; Zvarova et al., 2004). Spinal cord segments were sectioned entirely from rostral to caudal. Every third and sixth tissue section was then processed for PACAP-IR. Of these tissue sections, every first and fifth tissue section was then used for semi-quantitative analysis of PACAP-IR. We did not select sections based upon staining intensity and no sections were discarded from analysis because of low staining. Because stratification does not take the periodicity of the staining into account, it is random with respect to staining intensity. The following regions of spinal cord from both, experimental and control animals were analyzed: superficial, lateral dorsal horn (LDH) and medial dorsal horn (MDH), dorsal commissure (DCM) region, the region of sacral parasympathetic nucleus (SPN; L6, S1), the region of the intermediolateral cell column (IML; L2), Lissauer's tract (LT; L1, L2) and the region of lateral collateral pathway (LCP; L6, S1). Seven randomly chosen sections from each spinal segment examined were viewed with a 4× objective and captured through a video camera attachment to the microscope with exposure time, brightness and contrast being held constant. The image was converted into pixels on the computer monitor according to a gray scale that ranges in intensity from 0 (white) to 255 (black). The spinal cord section was centered in the field and a standard size square was overlaid on the areas of interest (LDH, MDH, DCM, SPN, IML, LT and LCP regions). The labeled area within the square was measured. Transmittance (t) was calculated as t=(gray level+1/256). Optical density (OD) was derived from OD=−log t. Comparisons among control and experimental groups were made using analysis of variance. When F ratios exceeded the critical value (p≦0.05), the Dunnett's test was used to compare the control mean with the experimental mean.

Figure Preparation

Digital images were obtained using a CCD camera (MagnaFire SP; Optronics; Optical Analysis Corp., Nashua, N.H.) and LG-3 frame grabber attached to an Olympus microscope (Optical Analysis Corp.). Exposure times, brightness and contrast were held constant when acquiring images from spinal intact and SCI animals processed and analyzed on the same day. Images were imported into Adobe Photoshop 7.0 (Adobe Systems Incorporated, San Jose, Calif.) where groups of images were assembled and labeled.

### Example 1

### PACAP6-38 Reduces Micturition Frequency with Cystitis

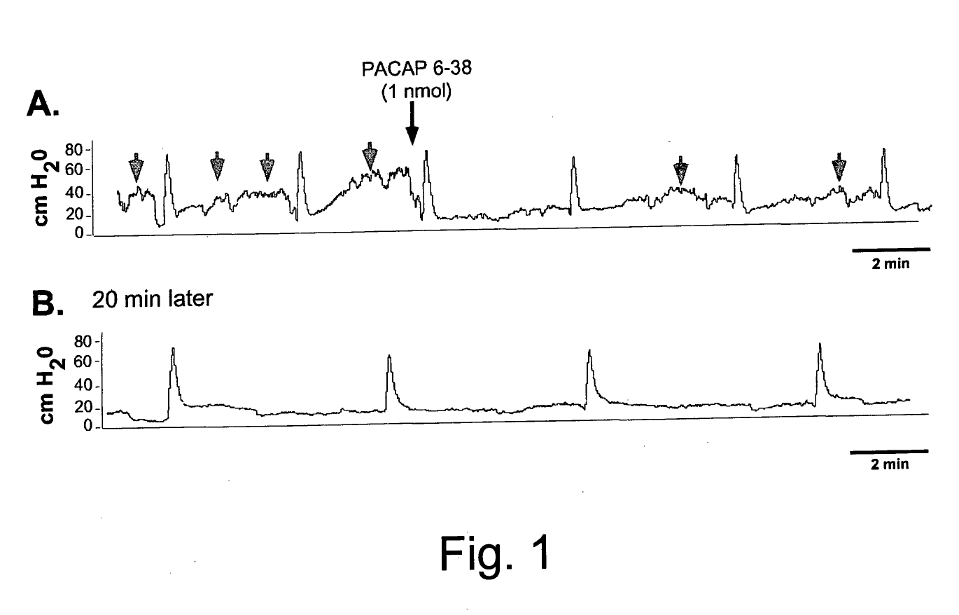

PACAP6-38 (1 nmol) delivered intrathecally (black arrow) reduced the appearance of non-voiding bladder contractions (gray arrows) and increased the intercontraction interval in a rat with chronic CYP-induced cystitis (FIG. 1). The drug effects persisted for at least 5 hours after administration.

### Example 2

### PACAP (6-38) Reduces Bladder Overactivity

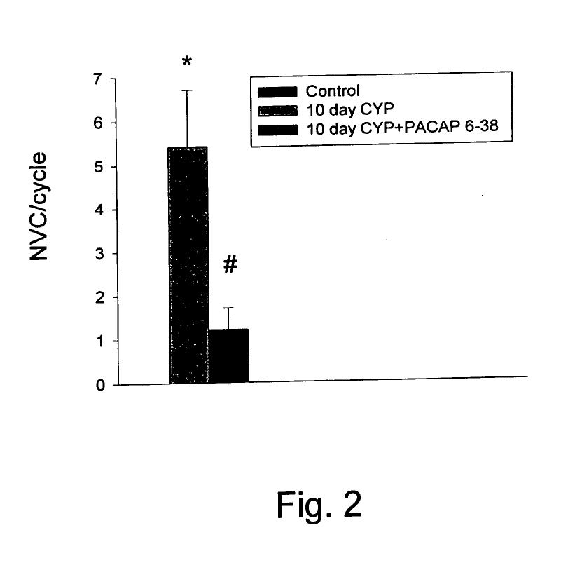

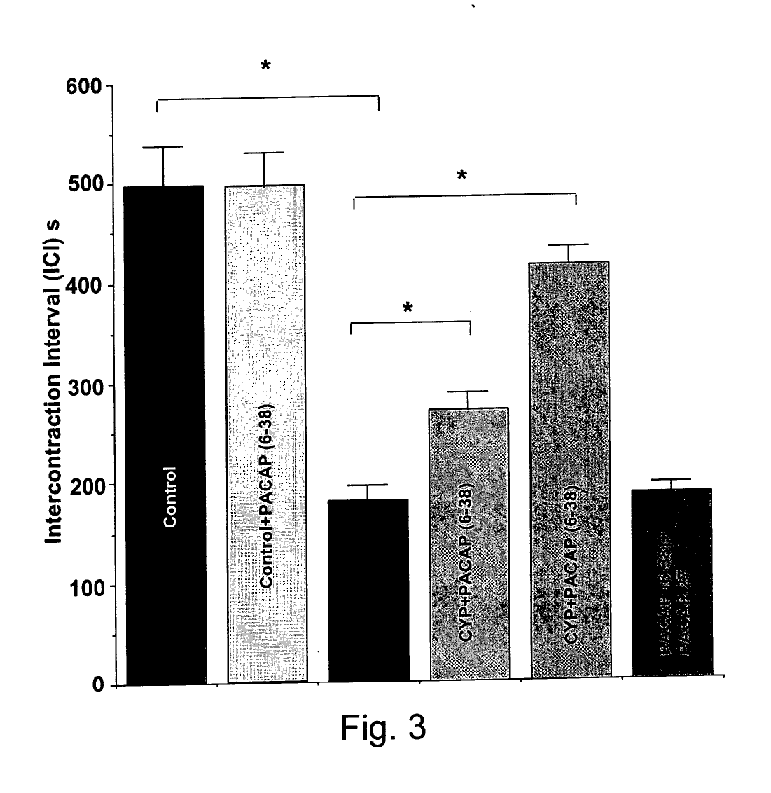

FIGS. 2 and 3 are summary figures for two parameters that were significantly affected by PACAP6-38 in rats treated chronically with CYP. Non-voiding bladder contractions per cycle (NVC/cycle) were significantly reduced compared to untreated rats with CYP-induced cystitis. Intercontraction interval was significantly lengthened with PACAP6-38 treatment (1 or 10 nmol). The change in intercontraction interval was reversed by treatment with agonist, PACAP. *, p<0.01; #, p<0.001.

### Example 3

### Intravesical PACAP6-38 Reduces Bladder Overactivity after Cystitis

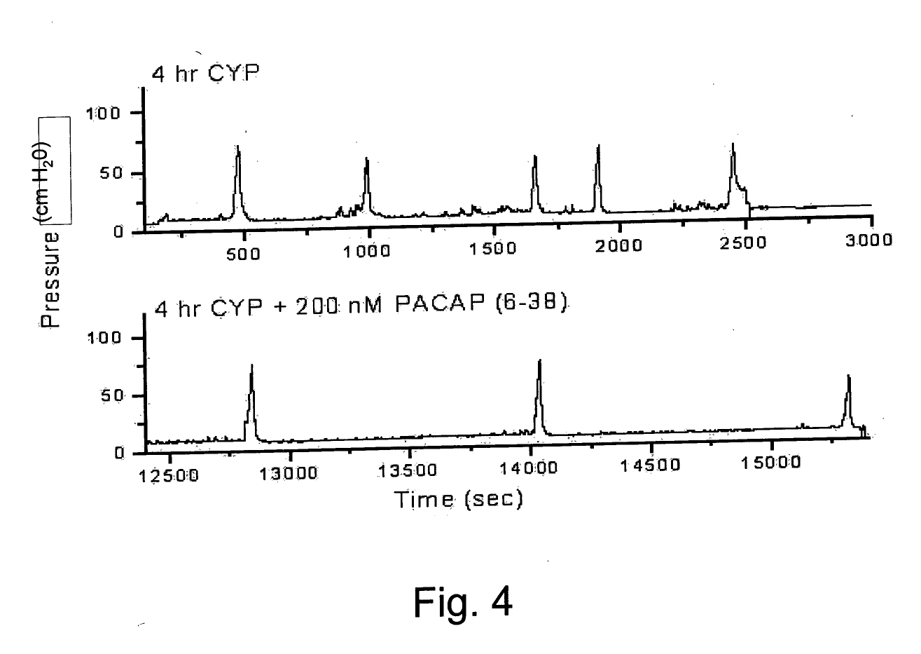

PACAP6-38 (200 nmol) delivered intravesically reduced the appearance of non-voiding bladder contractions (arrows) and increased the intercontraction interval in a rat with acute (4 hr) CYP-induced cystitis (FIG. 4). The drug effects persisted for at least 2 hours after administration.

### Example 4

### Intrathecal PACAP6-38 Reduces Micturition Pressures after SCI

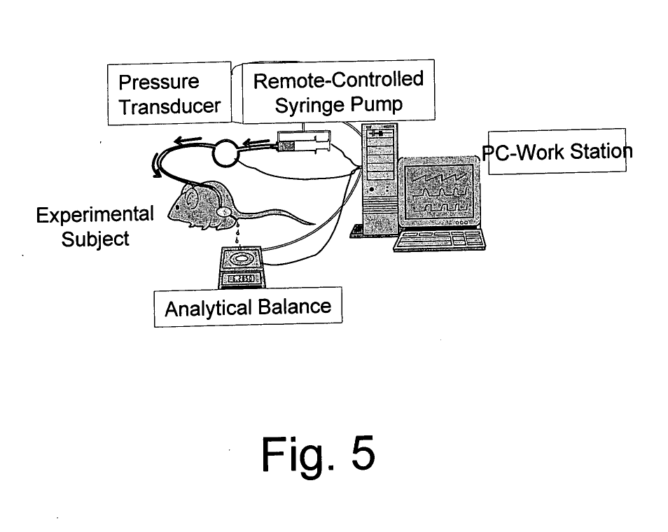

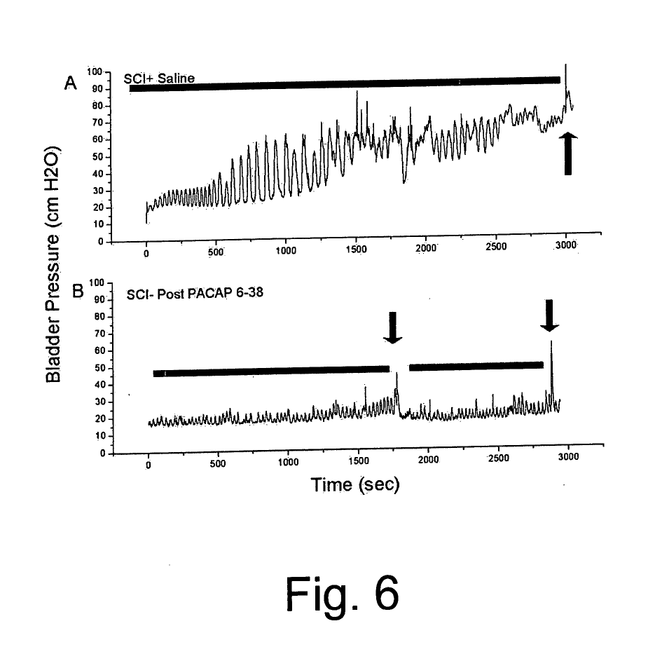

PACAP6-38 (1 nmol) delivered intrathecally reduced micturition pressures in a rat with chronic spinal cord injury (SCI) (FIGS. 5 and 6). The drug effects persisted for at least 5 hours after administration. Non-voiding bladder contractions were not eliminated by treatment but reduced in pressure.

### Example 5

### PACAP(6-38) Reduces Micturition Pressures

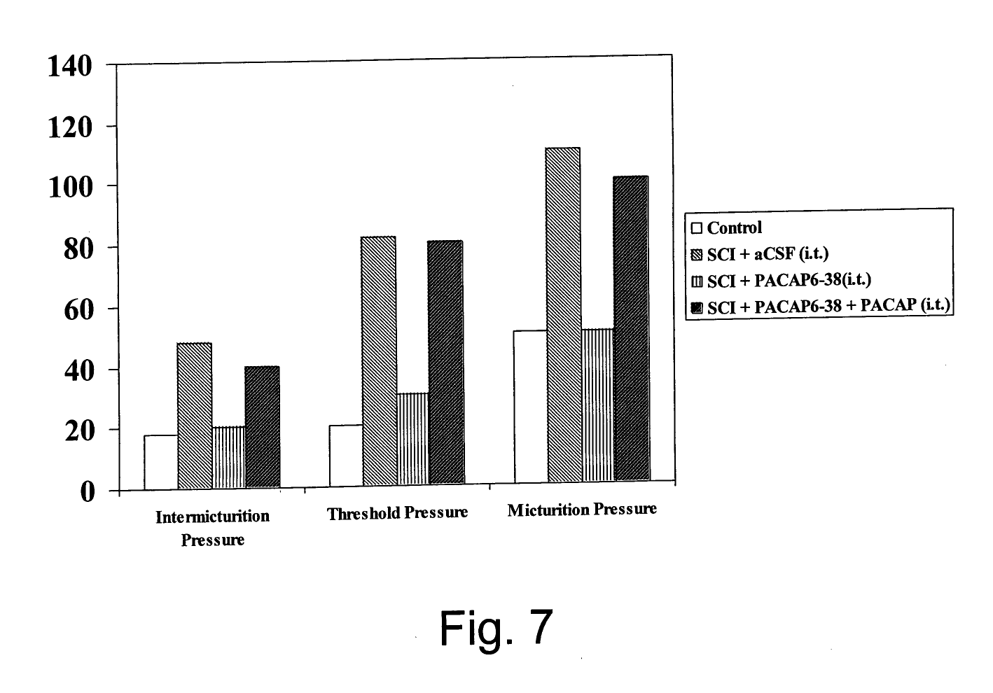

Intermicturition pressure, threshold pressure and micturition pressure were reduced by PACAP6-38 (1 nmol) treatment in spinal cord injured (SCI) rats compared to SCI rats treated with vehicle (artificial cerebrospinal fluid, aCSF). Intrathecal treatment with agonist, PACAP, blocked this effect. *, p<0.01 (FIG. 7).

### Example 6

### Comparison of Pituitary Adenylate Cyclase Activating Polypeptide (PACAP) Antisera

Staining with the monoclonal PACAP antibody resulted in intense PACAP-immunoreactivity (PACAP-IR) in cell bodies in the lumbosacral DRG whereas PACAP-IR in fibers in the spinal cord segments examined was less intense. Staining with the PACAP-27 antibody resulted in very weak PACAP-IR in the spinal cord segments and DRG examined. In contrast, staining with the PACAP-38 antibody resulted in intense PACAP-IR in fibers in the spinal segments examined. However, background staining in DRG was much greater with the PACAP-38 antibody compared to the monoclonal antibody. Differences in staining between PACAP-27 and PACAP-38 observed are consistent with previous demonstrations indicating that PACAP-38 is the prevailing form in various tissues including the brain and that the amount of PACAP-27 in the rat brain is negligible compared to PACAP-38 (Arimura et al., 1991; Masuo et al., 1993). Due to the reduced background staining observed with the use of the monoclonal antibody, quantification of DRG sections was based solely upon the monoclonal antibody staining. Intrepretation of PACAP-IR fiber staining in spinal cord segments and in the urinary bladder is based upon staining with the PACAP-38 antibody. These staining properties were identical to that from a previous study (Vizzard, 2000).

### Example 7

### PACAP-IR in Spinal Cord—Distribution and General Characteristics

In spinal intact animals in all segmental levels (L1, L2, L4-S1) examined, PACAP-IR was expressed in distinct regions of the rat spinal cord. Some PACAP-IR was unique to specific segmental levels (i.e., rostral lumbar, L1-L2 or lumbosacral spinal cord, L6-S1) and other staining was similar in all segmental levels (L1, L2, L4-S1) examined. In all segmental levels examined in spinal intact animals, PACAP-IR was expressed in the superficial dorsal horn, medial and lateral laminae I-II (data not shown). Very faint PACAP-IR was occasionally observed in the region of the dorsal commissure located dorsal to the central canal. In the lumbosacral (L6-S1) spinal cord, faint PACAP-IR was also expressed in the region of the sacral parasympathetic nucleus (SPN). Little if any PACAP-IR was observed in the ventral horn of any segmental level examined. PACAP-IR was expressed in nerve fibers in specific regions of the spinal cord and had a punctate staining quality. In the spinal cord, PACAP-IR was not expressed by neuronal cell bodies.

### Example 8

### PACAP-IR in the Lateral Collateral Pathway in the L6-S1 Spinal Cord

Faint PACAP-IR fiber staining in the L6-S1 spinal cord of spinal intact animals was apparent in a fiber bundle extending ventrally from Lissauer's tract in lamina I along the lateral edge of the dorsal horn into the dorsal part of the sacral parasympathetic nucleus (SPN, data not shown). The general location of the PACAP-IR bundle in lamina I and its selective segmental distribution resembles the central projections of visceral afferents in the pelvic nerve which have been labeled in the rat and cat by axonal transport of horseradish peroxidase and designated the lateral collateral pathway of Lissauer's tract (LCP) (Morgan et al., 1981; Steers et al., 1991; Steers et al., 1996; Steers and de Groat, 1988).

### Example 9

### Changes in PACAP-IR in the Spinal Cord after SCI

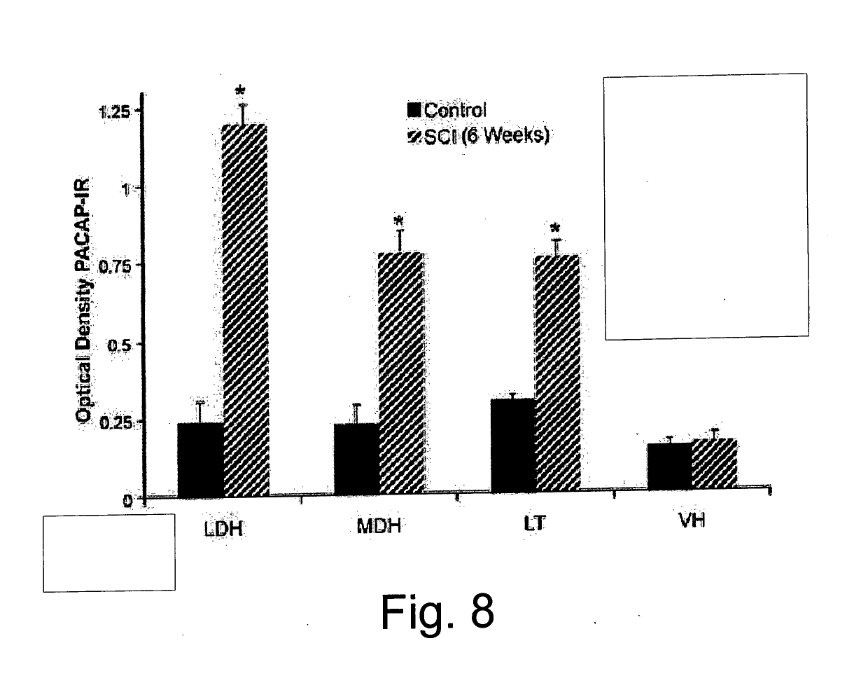

After SCI (6 weeks), the intensity and the overall distribution of the PACAP staining were increased in specific spinal cord segments and regions. After SCI (6 weeks), PACAP-IR increased in several regions in the rostral lumbar L1-L2 spinal cord compared to spinal intact rats (FIG. 8). The density of PACAP-IR was increased in the superficial laminae (I-II) of the dorsal horn having a denser distribution throughout the entire medial (3.1-fold increase) to lateral (5.0-fold increase) extent of the laminae (FIG. 8). Increased (2.8-fold increase) PACAP-IR fiber staining was also present after SCI in a small fiber bundle extending laterally from Lissauer's tract (LT) in lamina I into the dorsolateral funiculus (FIG. 8). No dramatic changes in PACAP-IR were observed in the region of the IML following SCI. Similar changes were observed in the L2 spinal segment after SCI.

### Example 10

### Lumbosacral Spinal Cord (L4-S1)

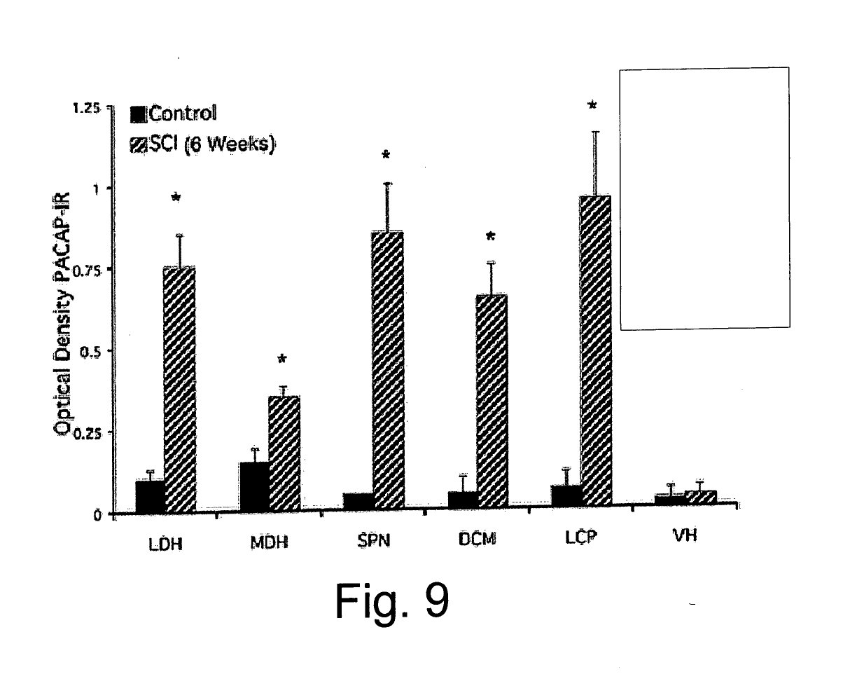

PACAP-IR was unchanged in the L4-L5 segments after SCI in any region examined: dorsal horn, dorsal commissure or lateral horn regions. In contrast, significant changes in PACAP-IR were detected in the L6-S1 spinal cord after SCI (FIG. 9). In the L6 spinal segment, PACAP-IR was dramatically increased in the dorsal horn (1.4-7.4-fold increase), dorsal commissure (7.0-fold increase), SPN (7.8-fold increase) and LCP (9.0-fold increase) (FIG. 9). Similarly, changes in PACAP-IR in the S1 segment were comparable to those in the L6 segment after SCI (data not shown). In some transverse sections of the L6-S1 spinal cord, PACAP-IR axons in the LCP terminated at the base of the dorsal horn whereas in others, they extended medially toward the central canal in distinct bundles through laminae V, VI and VII.

### Example 11

### PACAP-IR in Lumbosacral Dorsal Root Ganglia (DRG)

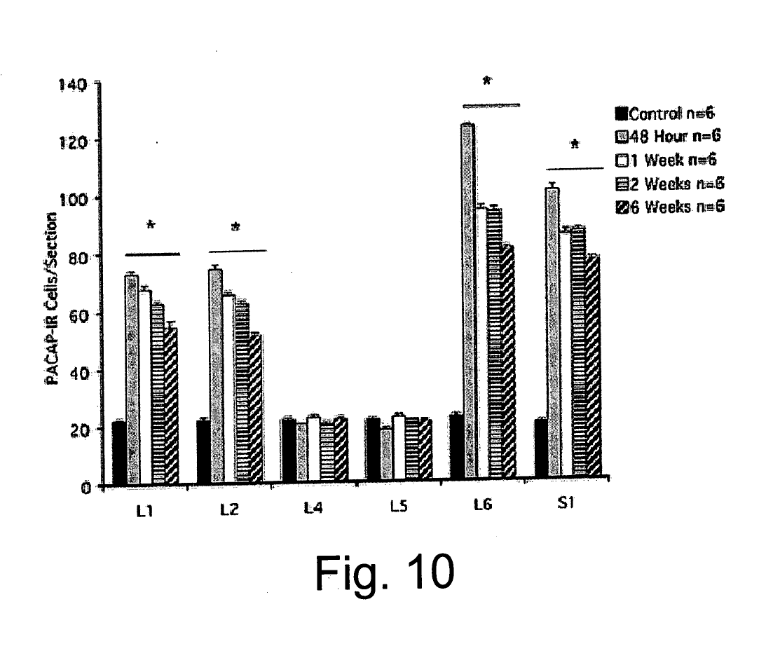

In contrast to PACAP-IR in the spinal cord, PACAP-IR in the DRG (L1-S1) was expressed by neuronal cell bodies and fibers throughout each DRG examined. In control animals, PACAP-IR was present in small numbers of cells in the L1-S1 DRG (FIG. 10). The number of PACAP-IR cells among the DRG examined was comparable (range 20-24 PACAP-IR cell profiles/section). After SCI of various durations (48 hr-6 weeks), PACAP-IR was significantly (p≦0.001) increased in the rostral lumbar (L1-L2) and lumbosacral (L6-S1) DRG (FIG. 10). Both small (16.8±3.5 μm) and medium (24.0±2.0 μm) sized DRG cells expressed PACAP-IR in spinal intact animals and SCI. PACAP-IR was occasionally observed in larger (≧30 μm) sized DRG cells. No change in numbers of cells expressing PACAP-IR was observed in the L4-L5 DRG after SCI of any duration (FIG. 10).

### Example 12

### PACAP-IR in Bladder Afferent Cells in Control Animals and after SCI

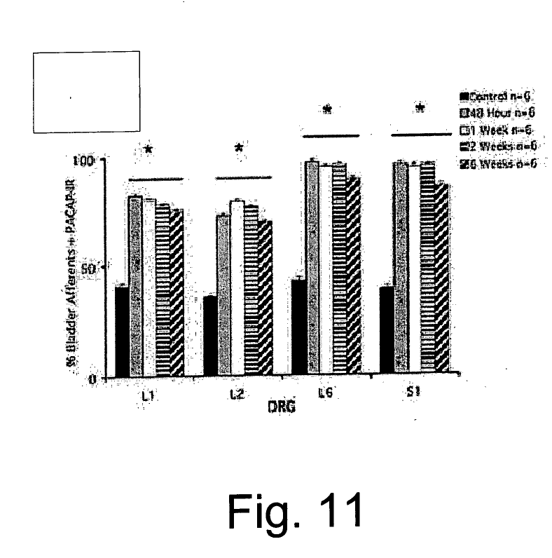

To determine if PACAP-IR was expressed in bladder afferent cells, Fastblue (FB) was injected into the urinary bladder to retrogradely label bladder afferent cells in the L1, L2, L6, S1 DRG. In spinal intact animals, approximately 45% of bladder afferent cells in the L6-S1 DRG exhibited PACAP-IR (FIG. 11). A similar percentage (40%) of bladder afferent cells in rostral lumbar DRG (L1-L2) of control animals also exhibited PACAP-IR. After SCI (6 weeks), the percentage of bladder afferent cells exhibiting PACAP-IR significantly (p≦0.001) increased in the L6 (88.8±2.2%) and S1 DRG (80.2±2.5%) and in the L1-L2 DRG (L1, 74.8±3.5%; L2, 69.5±3.2%) (FIG. 11). Not all bladder afferent cells expressed PACAP-IR either in spinal intact rats or after SCI nor were all PACAP-IR cells in the DRG accounted for by FB-labeled bladder afferent cells (FIG. 11). Increases in the percentage of bladder afferent cells expressing PACAP-IR after SCI were observed at the earliest time point after SCI (48 hours) and were maintained up to 6 weeks after SCI with only modest changes in the percentage of bladder afferent cells expressing PACAP (FIG. 11). The number of PACAP-IR cell profiles/section in DRG (L1-S1) examined from spinal intact animals and SCI animals with or without FB were not different (data not shown).

### Example 13

### PACAP-IR in the Detrusor Muscle and Urothelium in Control and SCI Rats

In spinal intact animals, PACAP-IR nerve fibers were present in the detrusor and urothelium whole-mounts (data not shown). In the detrusor, PACAP-IR was present in thick nerve trunks just dorsal to the point of ureter insertion (FIG. 7C). These fibers extended rostrally to the dome of the bladder but were of a finer caliber in the dome. PACAP-IR fibers were also present throughout the urothelium facing the detrusor. After 5 days or 3 weeks after SCI, PACAP-IR was decreased throughout the detrusor including the region dorsal to ureter insertion and in the urothelium. No PACAP-IR fibers were present on the adventitia portion of the detrusor. PACAP-IR nerve fibers in the detrusor or urothelium were colocalized with immunostaining for the capsaicin receptor (vanilloid receptor, VR1).

### Example 14

### siRNA Relieves Pelvic Floor Pain Syndrome, Overactive Bladder Disorder and Modulates Micturition Frequency

The effects of PACAP siRNA are examined on pelvic floor syndrome, overactive bladder disorder and micturition frequency. Animal models are used as described herein including cyclophosphamide (CYP)-induced cystitis-acute, intermediate or chronic, and chronic spinal cord injury.

Details of siRNA synthesis. Synthetic oligoribonucleotides with sequences that correspond to the PACAP receptor mRNA are designed as discussed herein, and are prepared using standard TOM-phosphoramidite chemistry (Xeragon AG) on an OligoPilot II synthesizer (Amersham Pharmacia Biotech) at 180 μmol scale. Phosphoramidites are dissolved in acetonitrile at 0.2 M concentration, mixed in a 1:1 ratio with a 0.2 M solution of benzimidazolium triflate in acetonitrile for coupling over 5 min. A first capping is made using standard capping reagents. Sulfurization is made by using a 0.05 M solution of N-ethyl,N-phenyl-5-amino-1,2,4-dithiazol-3-thione for 2 min. Oxidation is made by a 0.5 M t-butylhydroxyperoxide in dichloromethane for 2 min. A second capping is performed after oxidation or sulfurization. Oligonucleotides are detritylated for the following coupling by 2% dichloroacetic acid in dichloroethane. Upon completion of the sequences, the support-bound compounds are cleaved and base and phosphodiester deprotected as ‘Trityl-on’ material by a Methylamine solution (41% aqueous methylamine/33% ethanolic methylamine 1:1 v/v) at 35° C. for 6 h. Resulting suspensions are lyophilized to dryness. 2′-O-silyl groups are removed upon treatment with 1 M tetrabutylammonium fluoride (10 min at 50° C. and 6 h at 35° C.). The obtained crude solutions are directly purified by RP-HPLC. The purified detritylated compounds are analysed by capillary gel electrophoresis for purity and quantified by UV according to their extinction coefficient at 260 nM. Identity is checked by electrospray mass spectrometry.

siRNA annealing. For annealing of siRNA, 1 mM single strands are incubated in isotonic buffer (100 mM potassium acetate, 30 mM HEPES-KOH, 2 mM magnesium acetate, 26 mM NaCl, pH 7.4 at 37° C.) for 5 min at 90° C. followed by 1 h at 37° C.

Administration of PACAP siRNA oligonucleotides. siRNA oligonucleotides are administered intrathecally via an indwelling cannula as described herein. Briefly, rats are anesthetized, and an incision is made in the dorsal skin just lateral to the midline and ˜10 mm caudal to the ventral iliac spines. A sterile catheter (polyethylene PE10 tubing) is inserted via a guide cannula (20 gauge needle) and advanced 3 cm cranially in the intrathecal space approximately to the L1 level. The catheter, which is inserted subcutaneously in the left or right flank, was then connected to an osmotic minipump (Alzet) delivering the oligonucleotides or saline (1 μl/h, 7 d). The incision is closed with wound clips and dusted with antibiotic powder. Previous experiments have suggested 180 μg/d as a maximal tolerated dose. No evidence of neurotoxicity such as paralysis, vocalization, or anatomical damage to the spinal cord is noted at this dose. Delivery of oligonucleotides to the DRG cell bodies is initially confirmed using a fluorescently labeled oligonucleotides; an unlabelled version is used in all subsequent experiments. To assess whether intrathecal cannulation produced any nonspecific damage, a control group of naive animals that are cannulated and received saline is included. Additionally, contralateral thresholds are always measured, which would provide an indication of any nonspecific damage. The intrathecal dose of 400 μg/day PACAP siRNA or 180 μg/day PACAP antisense oligonucleotide is continuously infused over the course of 6-7 days. Each animal is tested in a blind random order. Statistical significance of data are analysed with ANOVA followed by Tukey'sHSD test, *P<0.05, **P<0.005, ***P<0.001.

Molecular analysis. Spinal cord sections, detrusor and urothelium for both treated and control animals are processed for PACAP-IR studies as described herein. The animals treated with siRNA show decreased expression of PACAP receptor. The animals treated with siRNA show reduced or eliminated pelvic floor pain syndrome, reduced or eliminated overactive bladder disorder and/or decreased micturition frequency.

Spinal cord sections, detrusor and urothelium for both treated and control animals are are snap-frozen and RNA is isolated separately for each animal by crushing the frozen cells with mortar and pestle and syringing 10 times to fragment genomic DNA. The RNA is then purified with RNeasy Columns (Qiagen, Basel, Switzerland). 50 ng of each sample is analysed three times with the ABI PRISM™ 7700 Sequence Detector (PE Applied Biosystems, Foster City, Calif.). Values are then normalized to β-actin analysis done in parallel on the same samples. Statistics: ANOVA followed by Tukey's HSD test, *P<0.05.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The content of each patent and scientific publication, and any other reference listed herein, is hereby incorporated by reference in its entirety to the extent that it contains relevant technical information.

1. A method for treating pelvic floor pain syndrome in a subject, comprising administering to the subject an inhibitor of PACAP receptor activity in an amount effective to reduce or eliminate the pelvic floor pain syndrome.

2. The method of claim 1, wherein the pelvic floor pain syndrome is associated with a condition selected from the group consisting of: overactive bladder, interstitial cystitis, outlet obstruction, benign prostatic hyperplasia, detrusor smooth muscle, bladder afferents, neurogenic bladder, bacterial and non-bacterial infections, and incontinence.

3. (canceled)

4. (canceled)

5. A method for modulating micturition frequency in a subject in need thereof, comprising administering to the subject an inhibitor of PACAP receptor activity in an amount effective to reduce the micturition frequency.

6. The method of claim 1, wherein the inhibitor of PACAP receptor activity is a monoclonal antibody.

7. The method of claim 1, wherein the inhibitor of PACAP receptor activity is a PACAP antagonist.

8. The method of claim 7, wherein the PACAP antagonist is selected from the list comprised of: PACAP (6-38), PACAP (6-27) or a mixture thereof.

9. The method of claim 1, wherein the inhibitor of PACAP receptor activity is an isolated short RNA nucleotide that has sequence correspondence to PACAP receptor mRNA and mediates RNA interference by directing cleavage of the PACAP receptor mRNA to which it corresponds.

10. The method of claim 1, wherein the inhibitor of PACAP receptor activity is an isolated short RNA nucleotide that has sequence correspondence to PACAP receptor mRNA and inactivates the PACAP receptor gene by transcriptional silencing.

11. The method of claim 1, wherein the inhibitor of PACAP receptor activity is an isolated short RNA nucleotide that has sequence correspondence to PACAP receptor mRNA and targets the PACAP receptor mRNA for degradation.

12. The method of claim 1, wherein the subject is a human.

13. The method of claim 1, wherein the inhibitor of PACAP receptor activity is administered in a pharmaceutically acceptable carrier.

14. The method of claim 13, wherein the pharmaceutically acceptable carrier is formulated for interstitial administration.

15. The composition of claim 13, wherein the inhibitor of PACAP receptor activity is formulated for administration by a needle.

16. The composition of claim 13, wherein the inhibitor of PACAP receptor activity is formulated for administration by intravesical catheter implant.

17. The composition of claim 13, wherein the inhibitor of PACAP receptor activity is formulated for administration by intrathecal catheter implant.

18. The method of claim 13, wherein the effective amount of the inhibitor of PACAP receptor activity is administered from once a day to once every other week.

19. A kit comprising packaging material and a formulation contained within the packaging material, wherein the formulation comprises an inhibitor of PACAP receptor activity, wherein the packaging material comprises a label or package insert that states that the formulation can be administered to a subject having pelvic floor syndrome in an amount, at a frequency, and for a duration effective to reduce or eliminate the pelvic floor syndrome.

20. (canceled)

21. (canceled)


SEQUENCE LISTING (10 sequences)

SEQ ID NO: 1 - protein, 33 aa, Artificial sequence
  Synthetic peptide
FTDSYSRYRKQMAVKKYLAAVLGKRYKQRVKNK

SEQ ID NO: 2 - protein, 22 aa, Artificial sequence
  Synthetic peptide
FTDSYSRYRKQMAVKKYLAAVL

SEQ ID NO: 3 - DNA, 1643 bp, Homo sapiens
cagccggccacacaggcgcggagacccggctcggcgcgcgctcctcggcgcacacgctccccatccccgcgccgcgcgggccgcggacttgcaggctgcgcgtcctttgcggagtctgccccggccccacggacaggccccgcagtgagcagcccagagacacattggggctgacctgccgctgctgtcagtgggaggccagtggtgctggccaagaagtgtcatggctggtgtcgtgcacgtttccctggctgctctcctcctgctgcctatggcccctgccatgcattctgactgcatcttcaagaaggagcaagccatgtgcctggagaagatccagagggccaatgagctgatgggcttcaatgattcctctccaggctgtcctgggatgtgggacaacatcacgtgttggaagcccgcccatgtgggtgagatggtcctggtcagctgccctgagctcttccgaatcttcaacccagaccaagtctgggagaccgaaaccattggagagtctgattttggtgacagtaactccttagatctctcagacatgggagtggtgagccggaactgcacggaggatggctggtcggaacccttccctcattactttgatgcctgtgggtttgatgaatatgaatctgagactggggaccaggattattactacctgtcagtgaaggccctctacacggttggctacagcacatccctcgtcaccctcaccactgccatggtcatcctttgtcgcttccggaagctgcactgcacacgcaacttcatccacatgaacctgtttgtgtcgttcatgctgagggcgatctccgtcttcatcaaagactggattctgtatgcggagcaggacagcaaccactgcttcatctccactgtggaatgtaaggccgtcatggttttcttccactactgtgttgtgtccaactacttctggctgttcatcgagggcctgtacctcttcactctgctggtggagaccttcttccctgaaaggagatacttctactggtacaccatcattggctgggggaccccaactgtgtgtgtgacagtgtgggctacgctgagactctactttgatgacacaggctgctgggatatgaatgacagcacagctctgtggtgggtgatcaaaggccctgtggttggctctatcatggttaactttgtgctttttattggcattatcgtcatccttgtgcagaaacttcagtctccagacatgggaggcaatgagtccagcatctacttgcgactggcccggtccaccctgctgctcatcccactattcggaatccactacacagtatttgccttctccccagagaatgtcagcaaaagggaaagactcgtgtttgagctggggctgggctccttccagggctttgtggtggctgttctctactgttttctgaatggtgaggtacaagcggagatcaagcgaaaatggcgaagctggaaggtgaaccgttacttcgctgtggacttcaagcaccgacacccgtctctggccagcagtggggtgaatgggggcacccagctctccatcctgagcaagagcagctcccaaatccgcatgtctggcctccctgctgacaatctggccacctgagccatgctcccct

SEQ ID NO: 4 - DNA, 3958 bp, Homo sapiens
gtgcattgagcgcgctccagctgccgggacggagggggcggcccccgcgctcgggcgctcggctacagctgcggggcccgaggtctccgcgcactcgctcccggcccatgctggaggcggcggaaccgcggggacctaggacggaggcggcgggcgctgggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtgcagagcttctgaggtctcaaacagaaaaacacaaagcctgcagtggcgtctgggacaacatcacgtgctggcggcctgccaatgtgggagagaccgtcacggtgccctgcccaaaagtcttcagcaatttttacagcaaagcaggaaacataagcaaaaactgtacgagtgacggatggtcagagacgttcccagatttcgtcgatgcctgtggctacagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccgcaacggctcggagggcgccctgcagttccaccgcggctcccgcgcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctggaattggagtcgtgttgtcattgactcgatttaaactccagcatttagataatcttgtgcaaaatgtgtttcagccgtatagtggatccacttttttttttttttttttttgagacggagtcttgctctgtcgcccaggctggagtgcagtggcctgatctctgctccctgcaagctccgcctcccgggttcacgccattctcctgcctcagcctcccatagctgggactacaggcgcccgccaacacgcctggctaattttttgtatttttagtagagacagggtttcaccatgttagccaggatggtctcgatctcctgacctcgtgatgggcccgcctcggcctcccaaagtgctgggattaaggcgtgagccactgcgcccggcccaagagaataggggagccaaggaggaaatgtggaaacgcagttgtgtggcccagcacgagcctgggcgaccaccgggtgacatccgtcccacatcagggcggcctcccaggtcccataagggtagccccctcatctgcaggacagagggaagccagtcagggcccccccgacgttaggaccaggagaaatcaacaggagggcagcccgtcctctctcttggggcgcccacccggcccggctgagccctgccccacccaactccacagggctgttttgcctccccacggaaggcgggctgaggagacaaccagatcaggagagcaaggtcatgaaggaggggacctctccacacaggtgttccgtgggaccctcagcagctctggctctgcctcaggaggtcacctgccgccctgtgggagccgcagagcctgacgctcagccccaggccagctgcggccaggcctgcgggcccctggtgatggggttacgtggggtgcgggatacagctgagtgggaaccggaaacctattctctttttaacaaaaataatcttaggataagaattattttaacaacatataaaactgtttcaagccctcctccccagagctggcgctcagcagccctagcggctgctccttcaggcgaagggtggtttgcagatgtggggagggtgtctggggacgttgctgagctggctgcagaagggtggggatatcagggcacagtctccatgtgtgtgccaagccctggcccccacagcgctcgatggacctcagcaagctgcccagccctggcccaggtgccccgactgtgggactcagttgttctgagcacatttgactccacttttcctttaaaaatgaatgtcttgttcctgtgcattggtggcatcacagaccccagctggggcgcgatgtcaaaggtcgggacagctgtgccgggaggcagccacagggaagctcacacatcctgtcagtgtcaccttggtttgcaaaacccatatccccggtaaaatgaggccggacagaggggctgttaggacagcaaagcagcagtgtccagagacccctcaatccccaaaggtccgcaccctgtcctgcacaccctgggccacgccggccacacccctctgctgcaacaagctcatccctggacttctgggagaatgaacccgaggttggtttggggagacaggtgaggcggttggatctacagaacaacccaccatttctgggggccgcagaggatccatcacagacggatactggggagtaaacggcccaggccaggtgcccaggaaaggacggctgagcatgtggagcgagagggaggcaggtggacgctgcagaccccaggttcagtgcggcccctcggctgttcctcccctgtagggtttggacagacccacccccagccttgcccagctttcaaaggacaaaagggagcatcccccacctactctcaggtttttgaggaaacaaagatttgtggtaactgaaggtgttgggtcagtggccaggtgccgacactgagctgtgacccagaggggacgctgaggaagtgggcgtgagtggacatgtcaggtggttaccaggcactggttgttgatggtcggtggttgggtgtgggcagtcatcagtcatcaggtgtgctcaggggacaatctcccctcaaccgcacatgtgccactgttcagcggagctgactggtttctcctggtagagggccggctgtatcctgacagatgcctggtgagcaggggaagcaggacccagtggtcaacaggtgtctttaactgtcattgtgtgtggaatgtcgcagactcctccacgtggcgggaatgagctgtgtaaatacttcaataaagcctgacttcacatctgcaaaaaaaaaaaaaaaaaa

SEQ ID NO: 5 - DNA, 2771 bp, Homo sapiens
ggccacaggccagcgccactctgccaggctcccggccatcgcccgcctggtgcgccgcccgccagctctttgcccgcgcggggccgcccgccgcgggctcagggcagaccatgcgcccgccaagtccgctgcccgcccgctggctatgcgtgctggcaggcgccctcgcctgggcccttgggccggcgggcggccaggcggccaggctgcaggaggagtgtgactatgtgcagatgatcgaggtgcagcacaagcagtgcctggaggaggcccagctggagaatgagacaataggctgcagcaagatgtgggacaacctcacctgctggccagccacccctcggggccaggtagttgtcttggcctgtcccctcatcttcaagctcttctcctccattcaaggccgcaatgtaagccgcagctgcaccgacgaaggctggacgcacctggagcctggcccgtaccccattgcctgtggtttggatgacaaggcagcgagtttggatgagcagcagaccatgttctacggttctgtgaagaccggctacaccattggctacggcctgtccctcgccacccttctggtcgccacagctatcctgagcctgttcaggaagctccactgcacgcggaactacatccacatgcacctcttcatatccttcatcctgagggctgccgctgtcttcatcaaagacttggccctcttcgacagcggggagtcggaccagtgctccgagggctcggtgggctgtaaggcagccatggtctttttccaatattgtgtcatggctaacttcttctggctgctggtggagggcctctacctgtacaccctgcttgccgtctccttcttctctgagcggaagtacttctgggggtacatactcatcggctggggggtacccagcacattcaccatggtgtggaccatcgccaggatccattttgaggattatgggtgctgggacaccatcaactcctcactgtggtggatcataaagggccccatcctcacctccatcttggtaaacttcatcctgtttatttgcatcatccgaatcctgcttcagaaactgcggcccccagatatcaggaagagtgacagcagtccatactcaaggctagccaggtccacactcctgctgatccccctgtttggagtacactacatcatgttcgccttctttccggacaattttaagcctgaagtgaagatggtctttgagctcgtcgtggggtctttccagggttttgtggtggctatcctctactgcttcctcaatggtgaggtgcaggcggagctgaggcggaagtggcggcgctggcacctgcagggcgtcctgggctggaaccccaaataccggcacccgtcgggaggcagcaacggcgccacgtgcagcacgcaggtttccatgctgacccgcgtcagcccaggtgcccgccgctcctccagcttccaagccgaagtctccctggtctgaccaccaggatcccaggggcccaaggcggcccctcccgccccttcccactcaccccggcagacgccggggacagaggcctgcccgggcgcggccagccccggccctgggctcggaggctgcccccggccccctggtctctggtccggacactcctagagaacgcagccctagagcctgcctggagcgtttctagcaagtgagagagatgggagctcctctcctggaggattgcaggtggaactcagtcattagactcctcctccaaaggccccctacgccaatcaagggcaaaaagtctacatactttcatcctgactctgccccctgctggctcttctgcccaattggaggaaagcaaccggtggatcctcaaacaacactggtgtgacctgagggcagaaaggttctgcccggggaaggtcaccagcaccaacaccacggtagtgcctgaaatttcaccattgctgtcaagttcctttgggttaagcattaccactcaggcatttgactgaagatgcagctcactaccctattctctctttacgcttagttatcagctttttaaagtgggttattctggagtttttgtttggagagcacacctatcttagtggttccccaccgaagtggactggcccctgggtcagtctggtgggaggacggtgcaacccaaggactgagggactctgaagcctctgggaaatgagaaggcagccaccagcgaatgctaggtctcggactaagcctacctgctctccaagtctcagtggcttcatctgtcaagtgggatctgtcacaccagccatacttatctctctgtgctgtggaagcaacaggaatcaagagctgccctccttgtccacccacctatgtgccaactgttgtaactaggctcagagatgtgcacccatgggctctgacagaaagcagatacctcaccctgctacacatacaggatttgaactcagatctgtctgataggaatgtgaaagcacggactcttactgctaacttttgtgtatcgtaaccagccagatcctcttggttatttgtttaccacttgtattattaatgccattatcctgaattccccttgccaccccaccctccctggcgtgtggctgaggaggcctccatctcatgtatcatctggataggagcctgctggtcacagcctcctctgtctgcccttcaccccagtggccactcagcttcctacccacacctctgccagaagatcccctcaggactgcaacaggcttgtgcaacaataaatgttggcttgga

SEQ ID NO: 6 - DNA, 1664 bp, Homo sapiens
agcccagagacacattggggctgacctgccgctgctgtcagtgggaggccagtggtgctggccaagaagtgtcatggctggtgtcgtgcacgtttccctggctgctcactgcggggcctgtccgtggggccggggcagactccgcaaaggacgcgcagcctgcaagtccgcggcccagagacacattggggctgacctgccgctgctgtcagtgggaggccagtggtgctggccaagaagtgtcatggctggtgtcgtgcacgtttccctggctgctctcctcctgctgcctatggcccctgccatgcattctgactgcatcttcaagaaggagcaagccatgtgcctggagaagatccagagggccaatgagctgatgggcttcaatgattcctctccaggctgtcctgggatgtgggacaacatcacgtgttggaagcccgcccatgtgggtgagatggtcctggtcagctgccctgagctcttccgaatcttcaacccagaccaagtctgggagaccgaaaccattggagagtctgattttggtgacagtaactccttagatctctcagacatgggagtggtgagccggaactgcacggaggatggctggtcggaacccttccctcattactttgatgcctgtgggtttgatgaatatgaatctgagactggggaccaggattattactacctgtcagtgaaggccctctacacggttggctacagcacatccctcgtcaccctcaccactgccatggtcatcctttgtcgcttccggaagctgcactgcacacgcaacttcatccacatgaacctgtttgtgtcgttcatgctgagggcgatctccgtcttcatcaaagactggattctgtatgcggagcaggacagcaaccactgcttcatctccactgtggaatgtaaggccgtcatggttttcttccactactgtgttgtgtccaactacttctggctgttcatcgagggcctgtacctcttcactctgctggtggagaccttcttccctgaaaggagatacttctactggtacaccatcattggctgggggaccccaactgtgtgtgtgacagtgtgggctacgctgagactctactttgatgacacaggctgctgggatatgaatgacagcacagctctgtggtgggtgatcaaaggccctgtggttggctctatcatggttaactttgtgctttttattggcattatcgtcatccttgtgcagaaacttcagtctccagacatgggaggcaatgagtccagcatctacttgcgactggcccggtccaccctgctgctcatcccactattcggaatccactacacagtatttgccttctccccagagaatgtcagcaaaagggaaagactcgtgtttgagctggggctgggctccttccagggctttgtggtggctgttctctactgttttctgaatggtgaggtacaagcggagatcaagcgaaaatggcgaagctggaaggtgaaccgttacttcgctgtggacttcaagcaccgacacccgtctctggccagcagtggggtgaatgggggcacccagctctccatcctgagcaagagcagctcccaaatccgcatgtctggcctccctgctgacaatctggccacctgagccatgctcccct

SEQ ID NO: 7 - DNA, 1817 bp, Homo sapiens
gggccgggacggagggggcggcccccgcgctcggggcgctcggctacagctgcggggcccgaggtctccgcgcactcgctcccggcccatgctggaggcgcggaaccgcggggacctaggacggaggcggcgggcgctggggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtgcagagcttctgaggtctcaaacagaaaaacacaaagcctgcagtggcgtctgggacaacatcacgtgctggcggcctgctaatgtgggagagaccgtcacggtgccctgcccaaaagtcttcagcaatttttacagcaaagcaggaaacataagcaaaaactgtacgagtgatggatggtcagagacgttcccagatttcgtcgatgcctgtggctatagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccacaacggctcggagggcgccctgcagttccaccgcgcgtcccgagcccagtccttcctccaaacggagacctcggtcatctagccccacccctgcctgtcggacgcggcgggaggcccacggttcggggcttctgcggggctgagacgccggcttcctccttccagatgcccgagcaccgtgtcgggctggtcagcgcggtcctgactccgtcaagctggttgtccactaaaccccatacctggaattggagtcgtgttgtcattgactcaacttaaactccagcaccacgaccctgctgctatctcgcacctgaaacaagctaacatgactaacacccttaattccatccaccctcctctccctaggaggcctgcgcccgctaaccggctttttcgcaaatgggccattatcgaa

SEQ ID NO: 8 - DNA, 1640 bp, Homo sapiens
cgggacgagggggcggcccccgcgctcggggcgctcggctacagctgcggggcccgaggtctccgcgcactcgctcccggcccatgctggaggcggcggaacccgggggacctaggacggaggcggcgggcgctgggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtacagagcttctgaggtctcaaacagaaaaacacaaagcctgcagtggcgtctgggacaacatcacgtgctggcggcctgccaatgtgggagagaccgtcacggtgccctgcccaaaagtcttcagcaatttttacagcaaagcaggaaacataagcaaaaactgtacgagtgacggatggtcagagacgttcccagatttcgtcgatgcctgtggctacagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccacaacggctcggagggcgccctgcagttccaccgcgcgtcccgagcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctgg

SEQ ID NO: 9 - DNA, 28 bp, Artificial sequence
  Synthetic oligonucleotide
ccgctcgagcatatggccagactcctgc

SEQ ID NO: 10 - DNA, 27 bp, Artificial sequence
  Synthetic oligonucleotide
gcgctcgagcgacaggtagtaataatc